(19) United States
(12) Patent Application Publication
(10) Pub. No.: US 2025/0135109 A1
(43) Pub. Date: May 1, 2025

(54) MEDICATION FLUID DELIVERY DEVICE COMPONENT AND METHOD OF USE

(71) Applicant: LUNA HEALTH, INC., Wilmington, DE (US)

(72) Inventor: Jack David Pryor, Signal Mountain, TN (US)

(21) Appl. No.: 18/924,000

(22) Filed: Oct. 23, 2024

Related U.S. Application Data
(60) Provisional application No. 63/650,541, filed on May 22, 2024.
(60) Provisional application No. 63/545,792, filed on Oct. 26, 2023.

(51) Int. Cl. A61M 5/158; A61M 5/142

(52) U.S. Cl. CPC A61M 5/158; A61M 5/14248; A61M 2202/0486

(57) ABSTRACT

A medication fluid delivery device component and associated methods are provided. The delivery device component includes: a wearable dispensing unit having a cannula in fluid communication with a reservoir for delivery of a medication fluid from the reservoir via a tip of the cannula during use of the dispensing unit; and an interface disposed at least partially around the cannula on a delivery side of the wearable dispensing unit and the interface being configured to receive an end portion of a medication filling device for filling of the reservoir via the tip of the cannula. The delivery device component may include a detachable support for the wearable dispensing unit with at least a portion of the interface being provided on the detachable support. The portion of the interface on the detachable support provides a protective cover for the tip of the cannula.

### CROSS-REFERENCE TO RELATED APPLICATIONS

The application claims priority to U.S. Provisional Patent Application No. 63/545,792, filed on 26 Oct. 2023, and U.S. Provisional Patent Application No. 63/650,541, filed on 22 May 2024, the contents of which are each hereby incorporated by reference in their entirety herein.

### FIELD OF THE INVENTION

This disclosure relates to medication delivery devices for delivery of medication fluid to people or animals to treat or prevent disease and associated delivery device component and methods of use.

### BACKGROUND OF THE INVENTION

Medication fluids for the treatment or prevention of disease in human or animal bodies often need to be input into the body at intervals. Many different devices for medication fluid input are known including syringes, intravenous drips, cannulas, etc. Regular administration of medication fluids outside of a medical setting has a number of challenges to ensure safe and appropriate dosing.

One form of medication fluid that requires administration outside of a medical setting is insulin administration. Millions of insulin-dependent people with diabetes around the world rely on multiple daily injections (MDI) to control their diabetes. Insulin pens are the most common insulin delivery method globally. A majority of insulin is currently delivered by ordinary insulin pens as opposed to people that use alternative delivery methods like smart insulin pens, insulin pumps, or automated insulin delivery systems.

People using insulin pens very often struggle to keep their blood glucose in a desired range overnight and may either have to wake up multiple times per night to administer insulin or risk hyper- and hypoglycemia. The result is disrupted sleep and other problems such as waking up tired, worried and groggy in the short term, and more profound health risks in the long term.

Automated Insulin Delivery (AID) systems such as insulin pumps with feedback from continuous glucose monitors (CGM) can control blood glucose levels continuously, including overnight. These systems are expensive, may be highly intrusive, and may require frequent infusion set changes. Many insulin pen users either choose to rely only on insulin pens or do not have access to insulin pumps.

Switching between insulin pens or other non-pump delivery during the day and short term use insulin pumps overnight is a current field of development.

The preceding discussion of the background to the invention is intended only to facilitate an understanding of the present invention. It should be appreciated that the discussion is not an acknowledgment or admission that any of the material referred to was part of the common general knowledge in the art as of the priority date of the application.

### SUMMARY OF THE INVENTION

According to a first aspect of the present invention there is provided a medication fluid delivery device component, comprising: a wearable dispensing unit having a cannula in fluid communication with a reservoir for delivery of a medication fluid from the reservoir via a tip of the cannula during use of the dispensing unit; and an interface disposed at least partially around the cannula on a delivery side of the wearable dispensing unit and the interface being configured to receive an end portion of a medication filling device for filling of the reservoir via the tip of the cannula.

In one embodiment, the interface may be configured to receive an insulin pen end of standard dimensions with a pen end having a diameter in the range of 9.48 mm to 9.52 mm as defined for receiving a double-ended pen needle assembly. In another embodiment, the interface may be configured to receive a vial top with the top having a diameter in the range of 19.5 mm to 22 mm.

The delivery device component may include a detachable support for the wearable dispensing unit with at least a portion of the interface being provided on the detachable support and the portion of the interface on the detachable support providing a protective cover for the tip of the cannula. The at least a portion of the interface may be a receiving collar surrounding an opening provided in the detachable support, such that the protective cover surrounds the cannula. The interface may be formed of the portion provided on a detachable support and a recess in the delivery side of the wearable dispensing unit surrounding the cannula. A depth of the recess may be a difference between a first length of the cannula required for filling and a second length of the cannula required for insertion into a body.

The delivery device component may include an attaching arrangement for securing the detachable support to the wearable dispensing unit. The attaching arrangement may be disposed at the interface and may be configured to have latches that are prevented from release by the end portion of the medication filling device when inserted in the interface. The attaching arrangement may be configured to be released automatically by interaction with a drive unit when the drive unit is attached to the wearable dispensing unit.

The attaching arrangement of the detachable support at the interface may include at least one resilient portion configured to press against and stabilize the medication filling device when inserted in the interface. There may be two opposing resilient portions at the interface and the resilient portions may together be configured to grip the medication filling device such that the detachable support remains attached to the medication filling device when removed from packaging.

The detachable support may include an engaging protrusion for engaging with a plunger of the wearable dispensing unit to provide a restriction on a movement of the plunger.

The wearable dispensing unit may include an alignment protrusion configured for alignment with a drive unit when attached to the wearable dispensing unit.

The delivery device component may include an adhesive arrangement including a skin adhesive member disposed on the delivery side of the wearable dispensing unit providing a skin adhesive surface configured to be exposed when the detachable support is removed. The skin adhesive member may include a skirt portion that is flexible with respect to the wearable dispensing unit to provide flexible attachment to a user's skin. The skirt may be within a footprint of the delivery side of the dispensing unit or may extend from the dispensing unit. The skin adhesive member may within a footprint of the delivery side of the wearable dispensing unit on two opposing sides and may extend beyond the footprint at one or both opposing ends.

The skin adhesive member may include a backing layer configured to cover the skin adhesive surface and the backing layer may have at least one attachment portion for attachment to the detachable support and may be configured such that removal of the wearable dispensing unit from the detachable support peels the backing layer from the skin adhesive surface. The backing layer may be a butterfly liner formed of two parts, each part including an adhesive covering portion and an attachment portion, wherein one or both of the attachment portions are folded back on the adhesive covering portions. The detachable support may include a liner anchor member extending from a device facing surface of the detachable support with the liner anchor member configured to engage an attachment portion of the backing layer. The device facing surface of the detachable support may have a recessed area accommodating the liner anchor member. The attachment portion or portions may include at least one slit configured for attachment to an anchor member, wherein the slit is in-line with a direction of removal of the backing layer.

The adhesive arrangement may include a device adhesive layer attached to a device facing surface of the skin adhesive member, wherein the device adhesive layer is formed of a pressure sensitive adhesive material and extends to opposing side edges of the skin adhesive member. The skirt portion of the skin adhesive member may have opposing side skirts that are within the footprint and the device adhesive layer may extend to the side skirts. The detachable support may be configured to apply pressure to the area of the device adhesive layer excluding areas of the side skirts to which the device adhesive layer extends. The dispensing unit may include side wings that are configured to not stick to the device adhesive layer at the areas of the side skirts. The dispensing unit may include side wings that are configured to flex with the side skirts.

The wearable dispensing unit may include a plunger for movement within the reservoir with the plunger having an interference fit with an internal surface of the reservoir removing a need for a sealing member. Alternatively, the wearable dispensing unit may include a plunger for movement within the reservoir with the plunger having a full-face seal that covers a front end of the plunger and formed of an elastomeric material. The full-face seal may conform to a shape of a front of the reservoir to reduce a dead volume in the reservoir. The plunger may include a curved rear face to minimize backlash in cooperation with the full-face seal.

The delivery device component may include a surrounding packaging of the wearable dispensing unit, wherein a part of the surrounding packaging provides a stable tray during a filling of the wearable dispensing unit. The delivery device component may include a surrounding packaging of the wearable dispensing unit, wherein at least part of the surrounding packaging is integrated with the detachable support.

The wearable dispensing unit may include a plunger operating member for retraction of a plunger of the wearable dispensing unit during filling from a vial.

According to another aspect of the present invention there is provided a medication fluid delivery device including a medication fluid delivery device component according to the first aspect, and a reusable drive unit for connection to the wearable dispensing unit for driving the delivery of the medication fluid from the reservoir through the cannula. The drive unit may include a form having opposing sides of a depth for gripping by a human finger and thumb.

According to a further aspect of the present invention there is provided a method of use of a medication fluid delivery device, comprising: filing a wearable dispensing unit through a tip of a cannula, wherein the cannula is in fluid communication with a reservoir for delivery of a medication fluid from the reservoir via the tip of the cannula during use of the dispensing unit.

The method may include positioning a medication filling device in an interface disposed at least partially around the cannula on a delivery side of the wearable dispensing unit such that the cannula is inserted into the medication filling device.

In one embodiment, the medication filling device may be an insulin pen and the method may include dispensing the medication from the insulin pen via the cannula to the reservoir.

In another embodiment, the medication filling device is a vial and the method may include retracting a plunger of the wearable dispensing unit to draw the medication from the vial via the cannula to the reservoir. Retracting the plunger may be via manual operation of the plunger. Alternatively, retracting the plunger may be via an automated activation of the plunger by attachment of a drive unit to the wearable dispensing unit.

Where at least a portion of the interface may be provided on a detachable support for the wearable dispensing unit, the method may include attaching a drive unit to the wearable dispensing unit thereby automatically releasing the detachable support from the wearable dispensing unit. Automatically releasing the detachable support from the wearable dispensing unit may be caused by the drive unit engaging with an attaching arrangement attaching the detachable support to the wearable dispensing unit. Releasing the detachable support from the wearable dispensing unit may expose an adhesive surface on a delivery side of the wearable dispensing unit and the tip of the cannula.

The method may include providing the wearable dispensing unit in a surrounding packaging, at least a portion of the surrounding packaging providing support for the wearable dispensing unit during filling of the wearable dispensing unit.

According to a further aspect of the present invention there is provided a detachable support for a wearable medicament delivery device, comprising: a protective base for the delivery device attached when packaged before use including: a central planar area configured to conform with a portion of an underside of the delivery device corresponding to an area of a device adhesive layer between a dispensing unit and a skin adhesive layer of the delivery device, wherein the planar member exerts pressure on the device adhesive material during packaging; and an adhesive protection removal arrangement configured to expose the skin adhesive surface of the skin adhesive layer of the delivery device on removal of the delivery device from the detachable support.

The adhesive protection removal arrangement may include one or more anchor members configured to anchor an attachment point of a backing liner of a skin adhesive surface of the delivery device, such that the backing liner is removed when the detachable support is removed from the delivery device prior to use. Alternatively, the adhesive protection removal arrangement may simply uncover the adhesive surface when the detachable support is removed.

The one or more anchor members may be provided in recessed portions of the detachable support. The detachable support may include at least a portion of an interface is configured to be at least partially around a cannula extending from a delivery side of the wearable dispensing unit and the interface being configured to receive an end portion of a medication filling device for filling the dispensing unit via the tip of the cannula.

According to another aspect of the present invention there is provided an adhesive arrangement for a medicament delivery device, comprising: a skin adhesive member configured to form a flexible skirt extending from an underside of the delivery device and having a skin adhesive surface; a device adhesive layer provided on an upper surface of skin adhesive member and for attachment to an underside of delivery device; and a backing liner covering the skin adhesive surface and configured for removal on release of the delivery device from a detachable support.

The backing liner may have one or more anchor openings for attachment to the detachable support prior to removal. The anchor openings may be slits provided during manufacture through the skin adhesive member and the backing liner.

The device adhesive layer may be a pressure sensitive adhesive layer that extends to opposing side edges of the skin adhesive member avoiding a need for separately die-cutting the pressure sensitive adhesive layer.

According to a further aspect of the present invention there is provided a method of manufacture of an adhesive arrangement of a medicament delivery device, including: providing a skin adhesive member and removing a native backing off an adhesive surface of the skin adhesive member; attaching a backing liner to the adhesive surface of the skin adhesive member; providing a device adhesive layer having dimensions covering a width of the skin adhesive member and less than a length of the skin adhesive member, removing a native backing off the device adhesive layer, and applying the device adhesive layer to a device-facing surface of the skin adhesive member; and cutting to shape a combined stack of the skin adhesive member, backing liner, and device adhesive layer.

The method may include cutting one or more anchor openings through at least one end portion of the combined stack.

Embodiments of the invention will now be described, by way of examples only, with reference to the accompanying drawings.

### BRIEF DESCRIPTION OF THE FIGURES

In the drawings:

FIGS. 1A and 1B are exploded top and bottom perspective views of an example embodiment of a delivery device assembly including a delivery device component suitable for filling from a pen;

FIGS. 2A and 2B are top and bottom perspective views of an example embodiment of a packaging tray holding the delivery device component;

FIG. 3A is an exploded perspective view of an example embodiment of a delivery device component including a dispensing unit and a detachable support;

FIG. 3B is a perspective view of an example embodiment of a delivery device including a dispensing unit and a drive unit;

FIGS. 4A to 4E are various views of an example embodiment of an interface arrangement of the delivery device component including a first attaching arrangement;

FIGS. 5A to 5G are a sequence of perspective views of an example embodiment of a delivery device component being filled from a pen;

FIG. 6 is a flow diagram showing a sequence of steps of an example embodiment of a method of using a delivery device component;

FIG. 7 is an exploded perspective views of another example embodiment of a delivery device assembly including a delivery device component suitable for filling from a vial;

FIGS. 8A to 8D are a sequence of perspective views of an example embodiment of a delivery device component being filled from a vial;

FIGS. 9A and 9B are a sequence of perspective views of another example embodiment of a delivery device component being filled from a vial;

FIGS. 10A and 10B are top and bottom perspective views of a further embodiment of the delivery device component including a second attaching arrangement;

FIG. 11 is a top perspective views of a further embodiment of the delivery device component including a third attaching arrangement;

FIGS. 12A and 12B are top and bottom perspective views of a further embodiment of the delivery device component including a fifth attaching arrangement;

FIG. 13 is a top perspective views of a further embodiment of the delivery device component including a fourth attaching arrangement;

FIG. 14 is a perspective underside view of an example embodiment of a delivery device showing an example of an adhesive arrangement;

FIGS. 15A to 15D are an exploded perspective view and side views of an example embodiment of a delivery device component showing another example of an adhesive arrangement;

FIGS. 16A to 16H are perspective views of a further example embodiment of an adhesive arrangement and base support;

FIGS. 17A and 17B are perspective views of a further example embodiment of a base support;

FIGS. 18A and 18B are various views of an example embodiment of a delivery device component showing a gripping arrangement.

FIG. 19 is a cross-section of an embodiment of the delivery device component including a plunger sealing arrangement; and

FIGS. 20A to 20D are perspective views of a further example embodiment of a plunger and sealing arrangement of the delivery device component.

### DETAILED DESCRIPTION

The disclosure describes a medication fluid delivery device for delivering a medication fluid to a human or animal body. In embodiments of the disclosure, the medication fluid is insulin and an insulin delivery device is provided for delivery of insulin percutaneously or subcutaneously. However, the delivery device is suitable for other forms of medication fluid that requires regular dosing percutaneously or subcutaneously.

An example wearable medication fluid delivery device is formed of two primary components: a dispensing unit that includes a fillable reservoir for the medication fluid and a cannula for delivering the fluid to a body; and a drive unit that includes a control unit for controlling a drive mechanism to cause the medication fluid to be delivered from the reservoir through the cannula. Such wearable medication fluid delivery devices may be referred to as pumps. When used to deliver insulin, such devices are often used in Automated Insulin Delivery (AID) systems. Wearable medication fluid delivery devices take various forms and may be designed to be worn for several days or for shorter durations such as during fasting periods only.

In some cases, the wearable medication fluid delivery device has a fillable reservoir. Conventionally, the reservoir has a filling port providing an inlet to the reservoir. Such known filling ports are provided on an upper side of the device on the opposite side of the device to the cannula outlet.

The described wearable medication fluid delivery device includes a cannula that is configured to be an inlet for the medication fluid to the reservoir as well as the delivery outlet. The cannula is provided in a fixed position and is configured to have a length suitable for filling as well as delivery. The cannula is suitable for filling from a medication filling device that has a septum for receiving the cannula. An example of such a medication filling device is a vial having a septum for receiving a needle. Another example of such a medication filling device is a medication delivery pen (such as an insulin delivery pen) that includes an end with a septum that is designed to receive a detachable double-ended pen needle (for example, as defined by ISO 11608-2).

The cannula is provided in the described wearable medication fluid delivery device that is suitable to use as a filling needle. This may be a steel cannula of at least 5.5 mm in length to make it suitable for filling. In the described embodiments, an interface is disposed at least partially around the cannula on a delivery side of the dispensing unit with the interface configured to receive an end portion of the medication filling device for filling of the reservoir via the tip of the cannula. The interface may be configured to accommodate a difference in the requirement of a length of needle for filling and a shorter length of needle for delivery to a user.

The two primary components of the wearable medication fluid delivery device of: the dispensing unit that includes the fillable reservoir and the cannula; and the drive unit that includes a control unit for controlling a drive mechanism to cause the medication fluid to be delivered from the reservoir through the cannula may be integrated as a single device. In other embodiments, the drive unit is detachable from the dispensing unit so that the drive unit may be reused multiple times with replaceable dispensing units. Such dispensing units may be configured to be filled from a medication filling device whether they are detachable or not. The description below focuses on dispensing units that are provided as replaceable components for the wearable medication fluid delivery device. However, it should be understood that the described interfaces may be adapted to be used with a wearable medication fluid delivery device having an integrated, non-detachable dispensing unit. In particular, the support member described below that provides at least a portion of the interface in some embodiments may be used with an integrated delivery device.

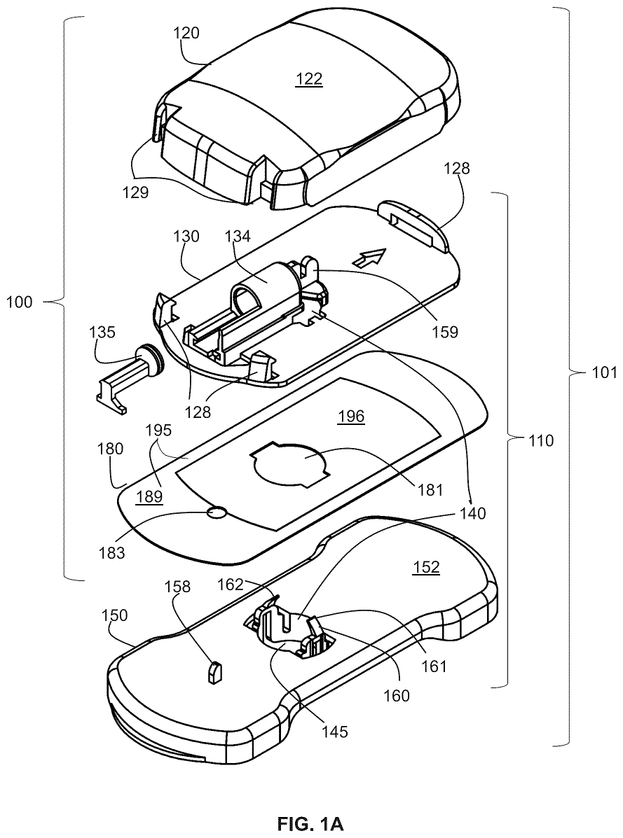

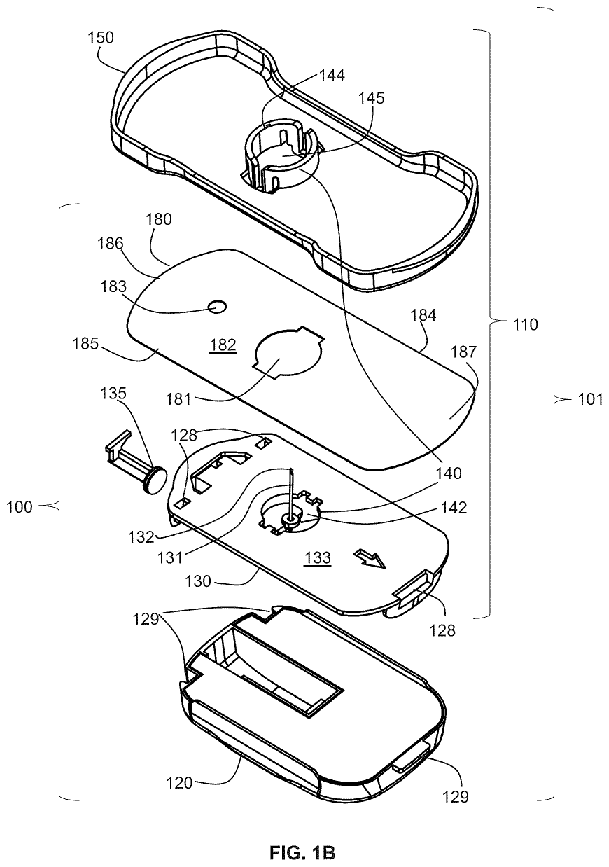

Referring to FIGS. 1A and 1B, top and bottom exploded perspective views are shown of an example embodiment of a wearable medication delivery device assembly (101). The delivery device assembly (101) includes a drive unit (120) that is attachable to a dispensing unit (130) to form a combined wearable delivery device (100) for wear on a user's body for delivery of the medication fluid to the body through the skin. The dispensing unit (130) has a delivery side (133) that supports a skin adhesive member (180) having a skin adhesive surface (182) for adhesion to the user's skin. The delivery device assembly (101) may also include a detachable support (150) provided with the dispensing unit (130) as a disposable delivery device component (110).

A single drive unit (120) may be used repeatedly with replacement dispensing units (130). Therefore, dispensing units (130) may be provided separately from the drive unit (120). The dispensing unit (130) includes a fillable reservoir (134) for the medication fluid with a plunger (135) for pushing the fluid from the reservoir through a cannula (131) for delivery of the fluid to a user's body. The cannula (131) has a base end at the reservoir (134) and a cannula tip (132) that is inserted into a user's skin. The cannula (131) may be a steel cannula of an example size of 30G to 32G (G is for Gauge).

The dispensing unit (130) may be provided as part of a delivery device component (110) that may be provided as a replacement component for a medication fluid delivery device (100). The delivery device component (110) includes the wearable dispensing unit (130) having the cannula (131) in fluid communication with the reservoir (134) for delivery of the medication fluid from the reservoir (134) via the tip (132) of the cannula (131) during use of the dispensing unit (130).

The delivery device component (110) includes an interface (140) disposed at least partially around the cannula (131) on the delivery side (133) of the dispensing unit (130). The interface (140) is configured to receive an end portion of a medication filling device for filling of the reservoir via the tip (132) of the cannula (131). In some embodiments, the interface (140) is configured to receive a medication delivery pen end of standard dimensions. The standard dimension may be as prescribed in ISO 11608-2 that provides standardization of a double-ended pen needle assembly and therefore the corresponding receiving pen assembly. According to ISO 11609-2, a standard pen hub diameter is 9.48 mm to 9.52 mm. In other embodiments, the interface (140) is configured to receive a vial top of standard dimensions. Vial dimensions are defined by ISO 8362-1 and may have a diameter at the interface of approximately 20 mm (approximately 19.5 mm to 22 mm). A vial commonly used for insulin is a 6R Crimp Neck Vial holding 10 ml.

The interface (140) may be integrated entirely into the dispensing unit (130) in the form of a recess (142) around the cannula (131). In another embodiment, the interface (140) may be entirely provided on a detachable support (150) that is provided with the dispensing unit (130) as part of the delivery device component (110). In a further embodiment, the interface (140) may have a portion provided by the recess (142) in the dispensing unit (130) and a portion provided on a detachable support (150) that is provided with the dispensing unit (130) as part of the delivery device component (110).

The detachable support (150) may have a shape and dimensions corresponding to the skin adhesive member (180) on the delivery side (133) of the dispensing unit (130). The detachable support (150) may be detached from the dispensing unit (130) when the dispensing unit (130) is ready to be attached to a user's body thereby exposing the skin adhesive member (180).

In an alternative embodiment, the detachable support (150) may be smaller than the dispensing unit (130) and may take the form of a collar arrangement providing at least a portion of the interface (140) and protection for the cannula (131) without an extending base portion. As mentioned above, in the case of an embodiment of a wearable medication delivery device that has an integrated dispensing unit and drive unit, a detachable support (150) may be provided detachable from the delivery side of the device.

The portion of the interface (140) provided on the detachable support (150) may provide a protective surround for the cannula tip (132) that projects from the delivery side (133) of the dispensing unit (130). The portion of the interface (140) provided on the detachable support (150) may include a receiving collar (144) provided on the detachable support (150) around an opening (145) positioned to surround the cannula (131) when the detachable support (150) is attached to the dispensing unit (130). The opening (145) may be dimensioned to receive an end of a medication filling device that includes a septum, such as a septum end of an insulin pen or a vial. The receiving collar (144) may extend around the opening (145) as a complete collar or may be a broken collar formed of two or more portions sufficient to guide the end of the medication filling device to a position in which the cannula (131) is inserted into the septum of the medication filling device. Due to this configuration, a medication filling device may be received in an underside of the detachable support (150), the opposite side to the side supporting the dispensing unit (130).

In one embodiment, the interface (140) is provided by a combination of the recess (142) around the cannula (131) in the delivery side. (133) of the dispensing unit (130) and the opening (145) and collar (144) extending from the detachable support (150). A depth of the recess (142) may be a difference between a first length of the cannula (131) required for filling from the medication filling device and a second length of the cannula (131) required for insertion into the user.

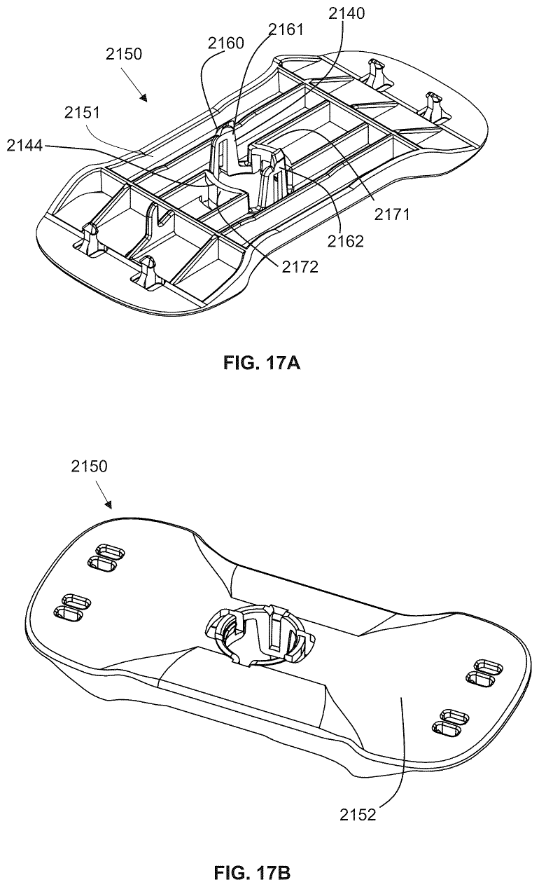

The delivery device component (110) may include an attaching arrangement (160) for securing the detachable support (150) to the dispensing unit (130) during filling of the dispensing unit (130). The attaching arrangement (160) may take various forms as described below. In one embodiment shown in FIGS. 1A and 1B the attaching arrangement (160) is disposed at the interface (140) and is configured to have latches (161, 162) that are prevented from release by the end portion of the medication filling device when inserted in the interface (140). In this embodiment, the attaching arrangement (160) is configured to be released automatically by interaction with a drive unit (120) when the drive unit (120) is attached to the wearable dispensing unit (130). The attaching arrangement may also include resilient portions that are shaped in conformity with part of the interface as shown in FIGS. 17A and 17B below.

The detachable support (150) may include an engaging protrusion (158) projecting from a device facing surface (152) for engaging with the plunger (135) of the dispensing unit (130) to provide a restriction on a movement of the plunger (135) when the detachable support (150) is attached to the dispensing unit (130). The restriction of the movement may provide a backstop for the plunger (135) to limit its movement and retain it within the reservoir (134) during filling. This may prevent the plunger (135) from being expelled from the dispensing unit (130) when the reservoir (134) is fully filled. A corresponding opening (183) may be provided in the adhesive member (180) to allow the engaging protrusion (158) to extend through the adhesive member (180).

The dispensing unit (130) includes a securing arrangement (128) corresponding to a securing arrangement (129) of the drive unit (120) to hold the drive unit (120) in place before attaching the delivery device (100) to the user. The dispensing unit (130) may include an alignment protrusion (159) configured for alignment with the drive unit (120) when attached to the wearable dispensing unit. The alignment protrusion (159) provides an axial alignment with a tight dimensional fit with a wall of the drive unit (120) to constrain the reservoir (134) axially in relation to the drive unit (120) so that it can precisely advance the plunger (135) during delivery. The alignment protrusion (159) aligns the reservoir (134) and the drive unit (120) so that the delivery dose is accurately dispensed.

An adhesive arrangement for adhering the wearable delivery device (100) to a user's skin is described. The adhesive arrangement has advantages for avoiding a user's contact with the tip of the cannular during application and reducing overall handling complexity. The adhesive arrangement includes an improved process for ease of manufacture. A form of the detachable support (150) for use in association with the adhesive arrangement is also described. A form of the dispensing unit (130) for cooperation with the adhesive arrangement is also described. These aspects associated with the adhesive arrangement may be provided for a medication delivery device that does not include the aspects of the interface and cannula filling described herein. In other words, the adhesive arrangement may be provided with other known types of patch pump or other medication delivery device that is attached to a user's skin.

The skin adhesive member (180) disposed on the delivery side (133) of the wearable dispensing unit (130) provides a skin adhesive surface (182). The skin adhesive member (180) may have an opening (181) corresponding to the interface (140). The skin adhesive member (180) may include a skirt portion (195) that is flexible with respect to the wearable dispensing unit (120) to provide flexible attachment to a user's skin. The skirt portion (195) may extend around an island of attachment of the skin adhesive member (180) to the dispensing unit (120). Alternatively, the skirt portion (195) may extend in one or more flaps from the dispensing unit (120).

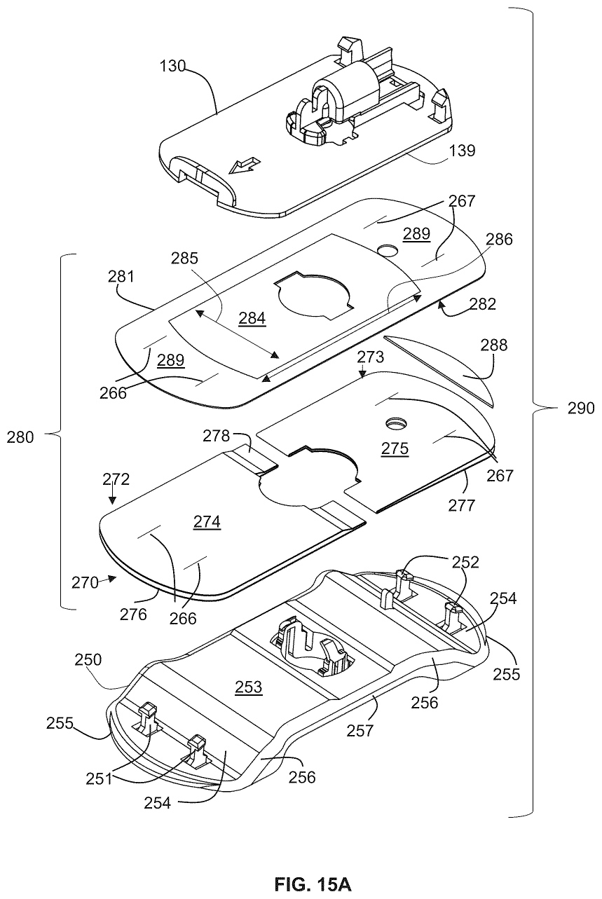

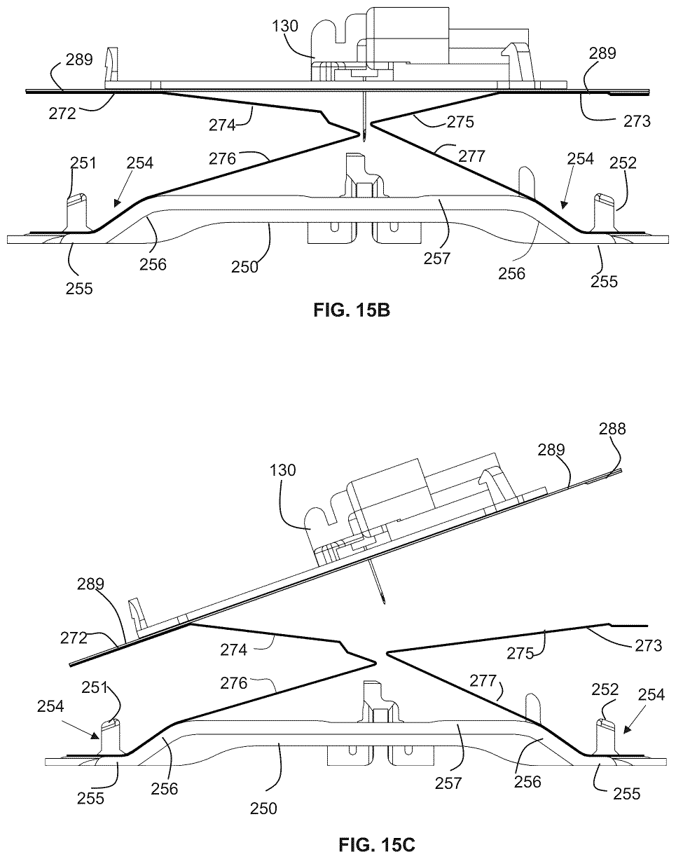

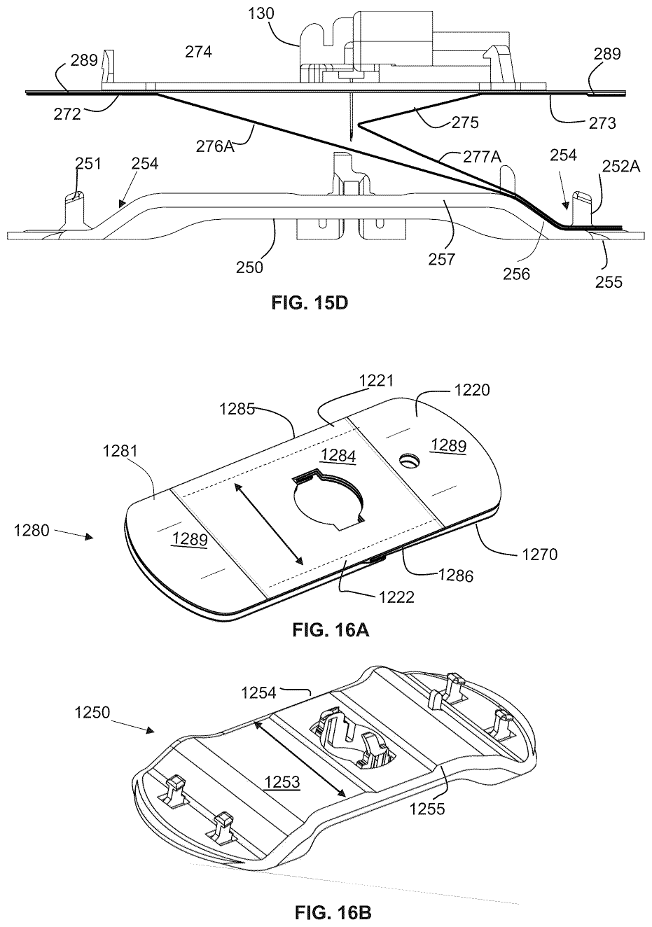

The skin adhesive member (180) may be within the footprint (139) of the dispensing unit (130) including a skirt portion (195), if provided. Alternatively, the skin adhesive member (180) may extend in at least one direction from the footprint (139) of the dispensing unit (130) as described further in relation to FIG. 15 below.

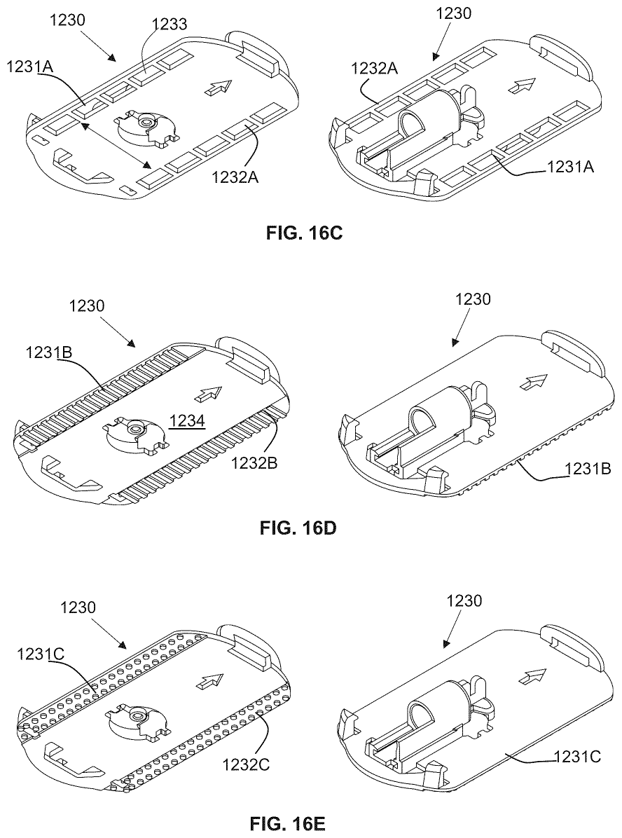

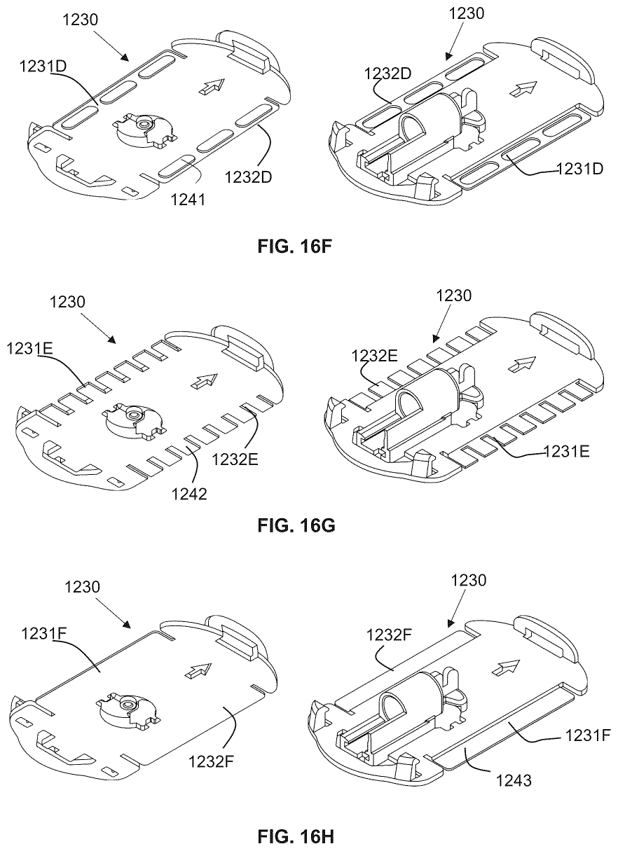

The skin adhesive member (180) has a skin adhesive surface (182) that may be configured to be exposed when the detachable support (150) is removed. The detachable support (150) may provide a protective cover for the skin adhesive surface (182) such that the skin adhesive surface (182) is exposed when the detachable support (150) is removed. In another embodiment, the skin adhesive member (180) may have a backing liner is provided between the adhesive surface (182) and the detachable support (150) and at least a portion of the backing liner may be attached to the detachable support (150) such that removal of the detachable support peels the backing liner away from the adhesive surface (182). An example embodiment is described further in relation to FIGS. 16A to 16D below. The arrangement of the removal of a backing liner may apply to wearable devices that do not include a skirt portion.

The adhesive arrangement may include a device adhesive layer (196) attached to a device facing surface (197) of the skin adhesive member (180). The device adhesive layer (196) may be formed of a pressure sensitive adhesive material and may extend to opposing side edges (184, 185) of the skin adhesive member (180). This has manufacturing advantages as it avoids the need to separately die-cut the device adhesive layer (196). Adaptations of the detachable support (150) and/or the dispensing unit (130) to accommodate this arrangement are described further in relation to FIGS. 17A to 17H below.

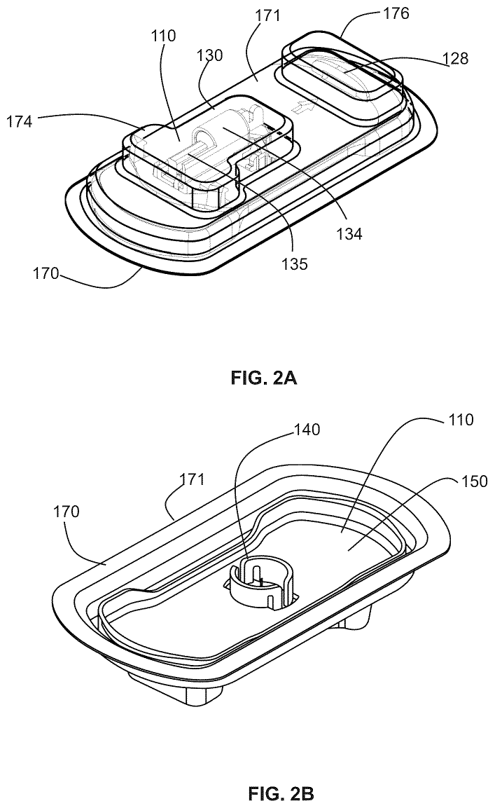

Referring to FIGS. 2A and 2B, top and bottom perspective views are shown of an example embodiment of a packaging (170) for the delivery device component (110). The packaging (170) provides a sealed enclosure for the delivery device component (110).

A part of the packaging (170) may provide a stable tray (171) for supporting the delivery device component (110) during a filling of the wearable dispensing unit (130). The tray (171) provides stability during filling of the dispensing unit (130). The tray (171) also provides a filling orientation by exposing the interface (140) to the user. The tray (171) further protects the delivery device component (110) before and during filling; in particular, the moving parts of the dispensing unit (130) are protected. The tray (171) may include a main recess (174) for the plunger (135) and reservoir (134) of the dispensing unit (130) with the recess (174) having a flat base so that the tray (171) rests securely on a surface. The tray (171) may also include a secondary recess (176) to accommodate a securing arrangement (128) of the dispensing unit (130) for attaching the dispensing unit (130) to the drive unit (120). In the illustrated embodiment, the securing arrangement (128) is a tab at one end of the dispensing unit (130) and the secondary recess (176) helps to provide additional support to the tray (171). The packaging (170) may include a removeable cover for the tray (171) such as a blister pack cover.

FIG. 2B shows an example of the delivery device component (110) as if the packaging (170) had just been opened by a user, ready to be filled. The detachable support (150) of the delivery device component (110) is exposed uppermost in this orientation. The packaging tray (171) supports the delivery device component (110) (e.g. in a user's hand or on a table) while the mediation filling device is inserted at the interface (140) for filling. The tray (171) protects the plunger (135) (e.g. from user's fingers or other objects that might interfere with it) while it slides back as the reservoir (134) is filled.

In another embodiment, the packaging of the delivery device component (110) is formed, at least in part by the detachable support (150) thereby minimizing the waste material. In this way, the surrounding packaging is integrated with the detachable support (150). This may include an attached cover portion and an adhesively attached sealing layer. The detachable support (150) may be configured to have an additional cover portion for enclosing the dispensing unit (130) with the cover portion having a snap fit lip around the detachable support (150). The additional cover may be shrink-wrapped to provide a sterile barrier for the dispensing unit (130) inside it. The detachable support (150) may have a seal layer glued across the interface side of the detachable support (150) that is peeled off before filling through the interface (140). After filling, the additional cover portion may be removed.

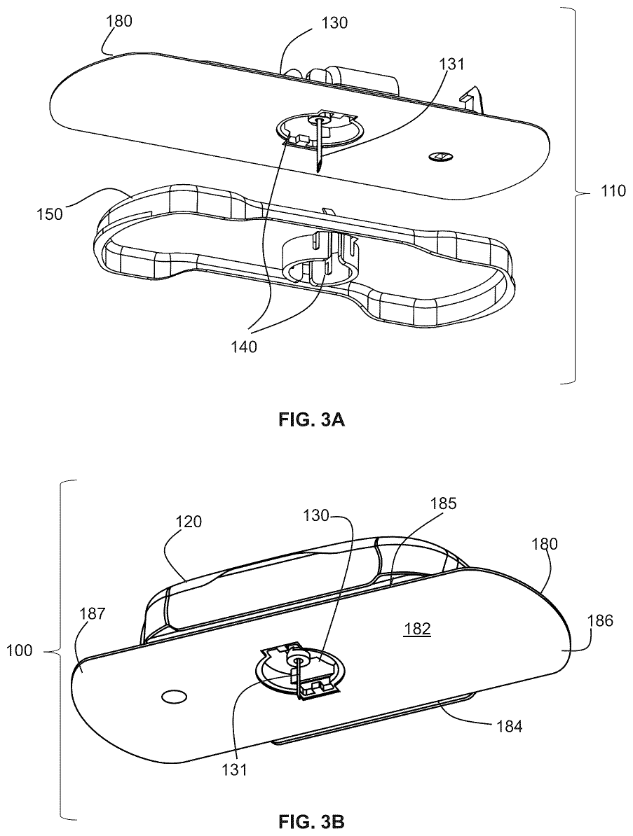

FIG. 3A is an exploded perspective view of an example embodiment of a delivery device component (110) including the dispensing unit (130), the skin adhesive member (180), and the detachable support (150). The delivery device component (110) may be provided in the packaging (170) of FIGS. 2A and 2B. FIG. 3A shows the interface (140) provided by the dispensing unit (130) and the detachable support (150) and passing through the adhesive member (180) and surrounding the cannula (131).

FIG. 3B is a perspective view of an example embodiment of the wearable form of the delivery device (100) including the dispensing unit (130) and the drive unit (120). In this form, the detachable support (150) has been removed prior to attachment to the user's body. This figures shows the skin adhesive member (180) ready for attachment to a user's body with the skin adhesive surface (182) exposed and the cannula (131) extending from the opening (181) in the adhesive member (180). The long sides (184, 185) of the adhesive member (180) may be within the sides of the drive unit (120) and therefore within the footprint of the delivery device (100). The ends (186, 187) of the adhesive member (180) may extend outwardly from the ends of the delivery device (100). In another embodiment, the long sides (184, 185) may match the footprint or extend a small distance (for example, 1 mm or less) beyond the footprint of the delivery device (100). This may maximize the adhesive area to the skin and prevent the plastic parts from directly contacting the skin whilst still not getting in the way of a user's finger and thumb gripping the delivery device (100).

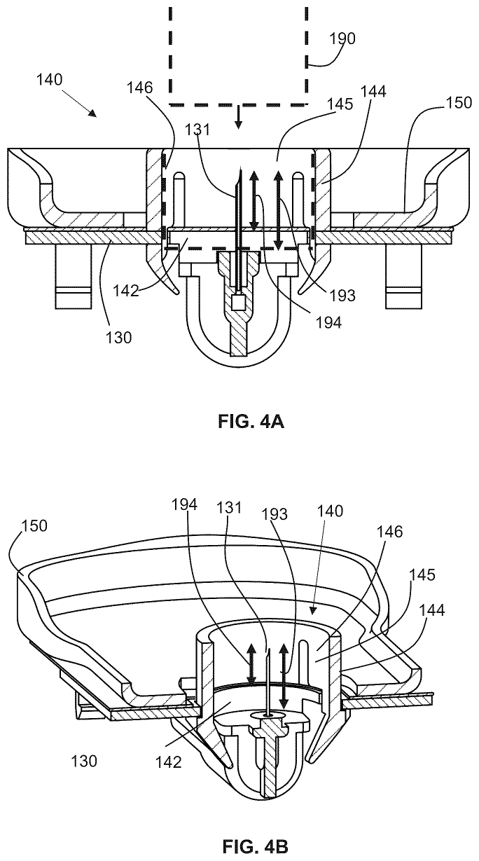

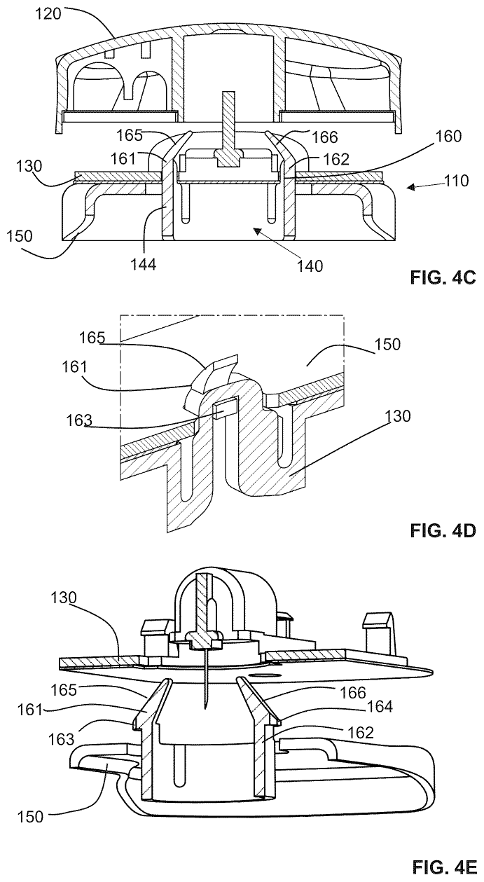

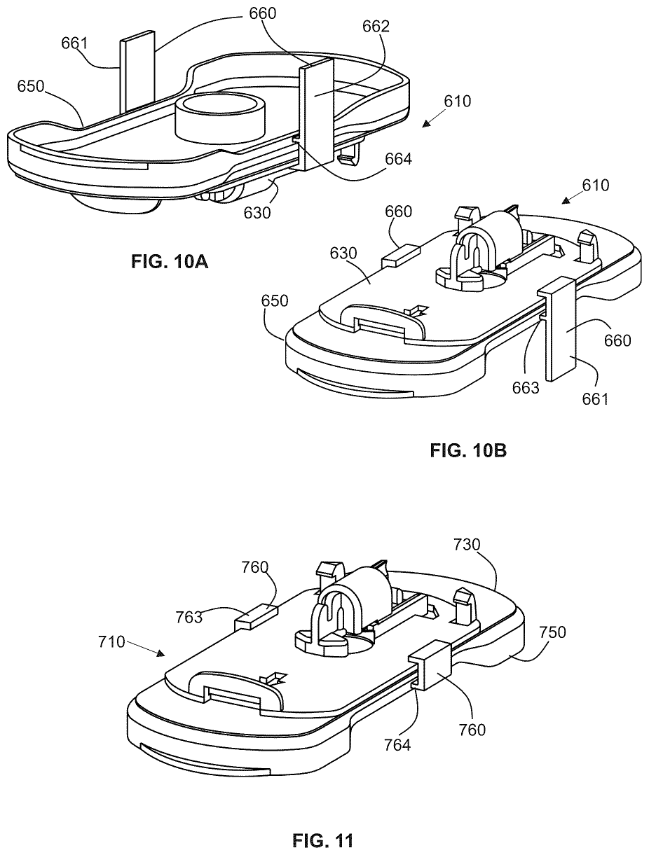

FIGS. 4A to 4E are various views of an example embodiment of an interface (140) of the delivery device component (110). In this example embodiment, a first attaching arrangement (160) is shown and described. However, the interface (140) of this embodiment may be used with any of the attaching arrangements as described with reference to FIGS. 10A, 10B, 11, 12A, 12B and 13. In this example embodiment, the dimensions of the interface (140) are suitable for filling by a medication filling device in the form of a pen delivery device. However, an equivalent interface (140) may be provided with a wider or narrower circumference for other forms of filling devices.

FIGS. 4A and 4B show a detail of the interface (140) provided at the cannula area of the dispensing unit (130) and at the detachable support (150) with the detachable support (150) engaged with the dispensing unit (130) as it is provided in the packaging and during filling by the user.

In this embodiment, the interface (140) is formed of a combination of a recess (142) in the dispensing unit (130) around the cannula (131) and an opening (145) with a surrounding collar (144) on the detachable support (150). The combination of the recess (142) and the opening (145) with the collar (144) make up a receiving cavity (146) that is suitable for standard interface for a medicament filling pen (190). Similarly, the dimensions could be adjusted to form a standard interface for a drug vial or similar. Medicament filling pens, such as insulin pens, have a septum designed for pen needles according to the standard ISO 11608-2.

In FIGS. 4A and 4B, the cannula (131) is shown centered in the receiving cavity (146). Having the receiving cavity (146) formed partly by the recess (142) in the delivery side of the dispensing unit (130) results in a length (193) of the cannula (131) in the receiving cavity (146) being suitable for filling from the medication filling pen whilst reducing the exposed length (194) of the cannula (131) from the dispensing unit (130). The recess (142) reduces the exposed length (194) by the depth of the recess (142).

For a medication delivery pen, the standard fitting of the pen requires a needle length of 4.7 to 7 mm to insert into a septum of the pen. This length is specified in ISO 11608-2 and refers to the amount of needle extending back into the septum of the pen, as measured from the needle hub face that bottoms-out on the end of the pen. The cartridge-end needle dimensions are defined in ISO 11608-2 Section 5.2.3 as l₂ of 5.7 mm-7 mm with a possible needle hub union h₁ of 0-1 mm, giving the full range of 4.7 mm-7 mm. A same length is therefore required for the cannula (131) to be inserted into a pen for filling the reservoir using a pen. The cannula (131) may have similar dimensions for the length (193) for insertion into a medicament filling pen as a medicament filling pen needle of 4.7 mm to 7 mm.

The delivery cannula (131) may require less length for insertion into a body than into the filling pen septum. For example, with a delivery device (100) intended for a short use of less than 24 hours, a 3.5 mm to 4.5 mm exposed length (194) extending from a base plane of the wearable delivery device (100) into a body may be suitable for most users. This exposed length (194) may be more specifically 3.8 mm to 4.2 mm and optimally 4 mm. The injection end length of insulin pen needles commonly ranges from 4 mm to 8 mm for insertion into the skin. In recent years there has been a shift away from longer needles as shorter ones are proven to be effective with less pain and tissue damage.

The difference between the filling length (193) and the exposed length (194) of the cannula (131) may be provided by the depth of the recess (142). This difference and therefore the depth of the recess (142) may be 0.2 mm to 3.5 mm for an exposed delivery length (194) of 3.5 mm to 4.5 mm. This range is obtained by the maximum depth of the recess of 3.5 mm being the maximum filling length of 7 mm less the minimum delivery length of 3.5 mm. The minimum depth of the recess is 0.2 mm being the minimum filling length (193) of 4.7 mm less the maximum exposed delivery length (194) of 4.5 mm. This may be rounded to zero where no recess is required as described in some embodiments herein. Embodiments with no recess may also suit longer delivery length cannulas as may be required by some users where the cannula has sufficiently length for insertion into the filling pen.

The interface (140) with the receiving cavity (146) provides for dimensional adaptation. The medicament filling device (190) is fitted to the interface (140) for filling and the interface (140) receives and provides guidance and stability to the filling device as the cannula (131) extends into the filling device to an appropriate depth controlled by the depth of the receiving cavity (146).

There may be a need to inject a cannula (131) deeper into the body than into a pen for filling. In such cases, the interface (140) may not include any recess, and the detachable support (150) may have an opening (145) and surrounding collar (144) that includes a spacing member to limit the pen filling depth of the interface (140). For example, the dispensing unit (130) may have 8 mm of cannular exposed from its base plane for injection into a body with the detachable support (150) having a spacer member standing-off the dispensing unit (130) by 2 mm so that only 6 mm of cannula (131) is insertable into a filling pen.

Medicament filling pens, such as insulin pens, have an end or hub supporting a septum according to the standard ISO 11608-2. The end may include a thread or other attachment arrangement to allow secure attachment of a needle. The interface (140) may include a corresponding attachment arrangement for securely receiving the filling pen. Alternatively, this is not required as the filling process does not require such securing and the interface (140) may be dimensioned to accommodate an attachment arrangement without engaging with it.

FIG. 4C shows the delivery device component (110) after filling that has been turned upside down so that it is resting on the detachable support (150) with the drive unit (120) being installed onto the dispensing unit (130). The drive unit (120) is about to engage the securing arrangement (128, 129 of FIGS. 1A and 1B) and disengage the attaching arrangement (160) between the dispensing unit (130) and the detachable support (150).

The attaching arrangement (160) is formed of two opposing latch arms (161, 162) that extend from the collar (144) of the interface (140) at an inward angle. The latch arms (161, 162) flex inwardly to provide a snap engagement (163) with the dispensing unit (130) as shown in FIG. 4D.

The latch arms (161, 162) have ramp faces (165, 166) that are pushed down and pinched together by a portion of the drive unit (120) when it is attached to the dispensing unit (130). The downward force on the ramp faces (165, 166) releases the snap engagement (163, 164) from the dispensing unit (130) and releases the detachable support (150) from the dispensing unit (130) as shown in FIG. 4E.

The ramp faces (165, 166) engage with the underside of the drive unit (120) to rotate the snap engagement (163, 164) inward and disengage it when the drive unit (120) is installed. Simultaneously, the dispensing unit (130) engages the securing arrangement (128, 129) with the drive unit (120), so that when the drive unit (120) is lifted up, the dispensing unit (130) releases from the detachable support (150) and stays attached to the drive unit (120), forming the entire wearable assembly to be placed onto the user's body.

The attaching arrangement (160) has the added benefit of preventing detachment of the detachable support (150) when the filling device is in the receiving cavity (146) by the filling device backing up the attaching arrangement (160) and preventing flexing of the latch arms (161, 162) and therefore preventing disengagement of the snap engagement (163, 164).

In this way, when the drive unit (120) is attached to the dispensing unit (130), the attaching arrangement (160) automatically releases the dispensing unit (130) from the detachable support (150). To attach the drive unit (120) to the dispensing unit (130), the user may angularly slide a tab end (129) of the securing arrangement on the control unit (120) into a corresponding slot (128) at an end of the dispensing unit (130). As the user presses down on the drive unit (120) to engage the snap securing arrangement (128, 129) at the opposite end of the drive unit (120) and the dispensing unit (130), the underside of the drive unit (120) pushes down on the ramp faces (165, 166) of the attaching arrangement (160). This force pushing down on the ramp faces (165, 166) pivots the snap engagement (163, 164) inwards and disengages from the dispensing unit (130).

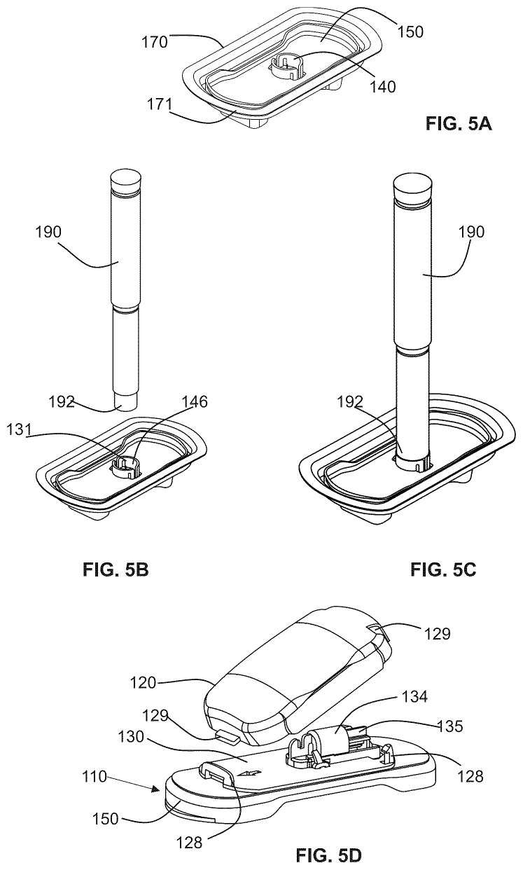

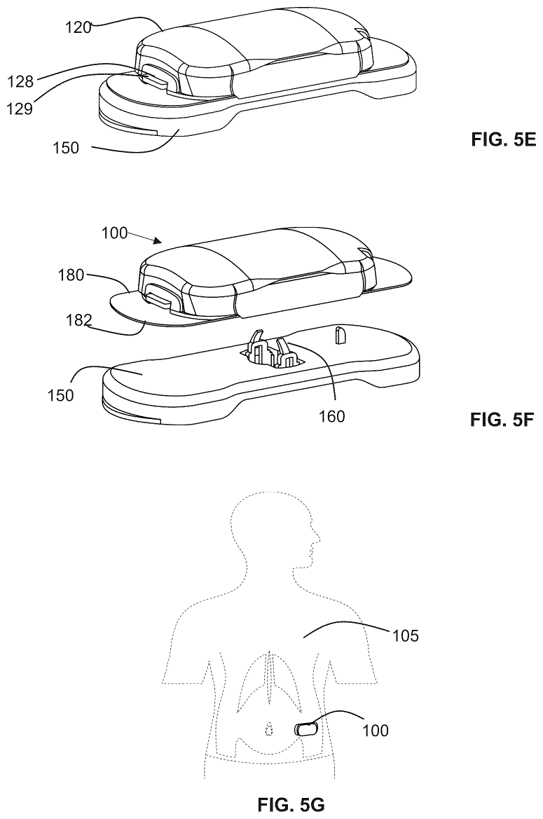

FIGS. 5A to 5G are a sequence of perspective views of an example embodiment of a delivery device component being filled from a medicament filling pen (190). FIG. 5A shows the packaging (170) open by removing a sterile barrier to leave the tray (171) that is stable on a supporting surface and presenting the interface (140) of the detachable support (150). FIGS. 5B and 5C show an end portion (192) of the pen (190) being inserted into the receiving cavity (146) of the interface (140).

When the pen (190) is inserted in the receiving cavity (146), the tip of the cannula (131) pierces a septum of the pen (190) and the medicament may be dispensed by actuation of the pen (190), filling the reservoir (134). In alternate embodiments described below, instead of a pen (190) the user may a medicament vial into an appropriately sized interface and may operate the plunger (135) to fill the reservoir (134).

The delivery device component (110) in the form of the combination of the dispensing unit (130) and the detachable support (150) is then taken out of tray (171) and turned upside down so that it sits with the detachable support (150) on the supporting surface. The drive unit (120) is attached as shown in FIG. 5D resulting in the arrangement of FIG. 5E. The securing arrangement (128, 129) couple the drive unit (120) to the dispensing unit (130) and the installation of the drive unit (120) disengages the attaching arrangement (160) to automatically decouple the dispensing unit (130) from the detachable support (150) in response to the attachment of the drive unit (120) as shown in FIG. 5F.

The drive unit (120) with the dispensing unit (130) attached and together forming the delivery device (100) is lifted off of the detachable support (150) exposing the adhesive surface and cannula.

A backing liner of the adhesive member (180) may be attached to the detachable support (150), such that once the dispensing unit (130) is lifted off of the detachable support (150), the backing is automatically peeled exposing the adhesive surface (182) and the delivery device (100) is ready to wear. The detachable support (150) may have affordances for holding it down during removal. The user's fingers are grasping the drive unit (120) on the sides, so the detachable support (150) may have grip features enabling it to be pulled away.

The delivery device (100) is placed onto the user's body (105) as shown in FIG. 5G with the cannula (131) piercing and penetrating through the skin until the adhesive is securely fastened on the skin surface. Manual insertion of the cannula (131) is described here; however, automatic or semi-automatic applicators may be used to hold and apply the delivery device (100) to aid with applying the force required to pierce and penetrate the skin. After wear, the delivery device (100) is removed from the user's body, and the drive unit (120) is manually unsnapped from the dispensing unit (130) and the dispensing unit (130) is discarded. The drive unit (120) is recharged for the next use.

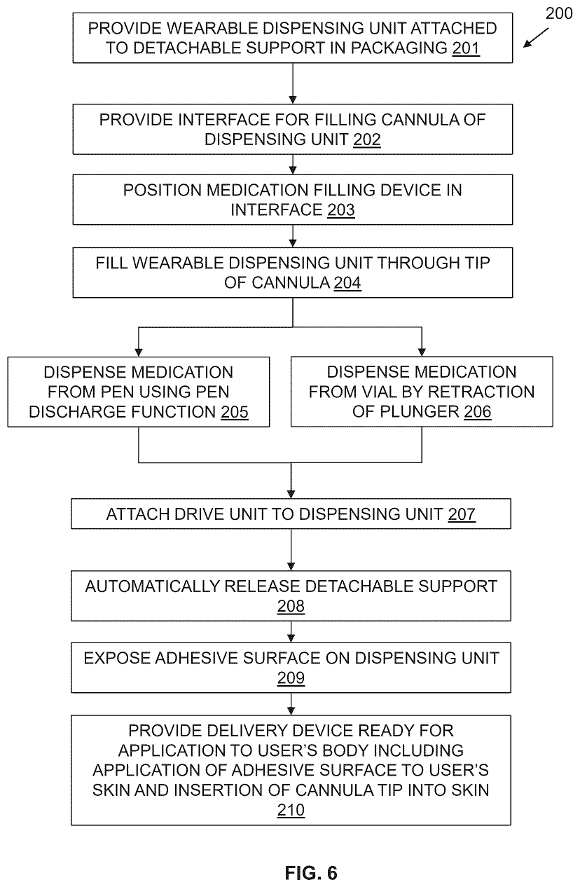

FIG. 6 is a flow diagram (200) showing a sequence of steps of an example embodiment of a method of using a delivery device component (110).

The method may provide (201) a wearable dispensing unit (130) in a surrounding packaging (170). At least a portion of the surrounding packaging (170) may provide support for the wearable dispensing unit (130) during filling of the wearable dispensing unit (130). The method may provide (201) the wearable dispensing unit (130) attached to a detachable support (150) that in combination provide (202) the delivery device component (110) with an interface (140) for filling the reservoir (134) of the dispensing unit (130) via the cannula (131) of the dispensing unit (120) that is also used to deliver the medicament to the user.

The method may position (203) a medication filling device in the interface (140) disposed at least partially around the cannula (131) on a delivery side (133) of the wearable dispensing unit (130) such that the cannula (131) is inserted into the medication filling device.

The method may fill (204) a wearable dispensing unit (130) through a tip (132) of the cannula (131) by insertion of the tip (132) into a septum of a medication filling device. The cannula (131) is in fluid communication within the dispensing unit (130) with the reservoir (134). The cannula (131) is the same cannula (131) that is used for delivery of the medication fluid from the reservoir (134) via the tip (132) of the cannula (131) during use of the dispensing unit (130). Filling through the same cannula that is used to dispense the medicament reduces any air in the cannula and reservoir thereby reducing priming requirements. By filling the reservoir (134) by the delivery cannula (131), medicament from the medication filling device fills the cannula (131) itself, thus minimal or no air remains inside the cannula (131) after filling. This simultaneously primes the cannula (131) with medication by virtue of and during the fill process.

When the medication filling device is a medicament pen, the method includes dispensing (205) the medication from the pen using the pen discharge function into the reservoir (134) via the cannula tip (132). When the medication filling device is a vial, the method includes dispensing (206) the medication from the vial by retracting the plunger (135) of the wearable dispensing unit (130) to draw the medication from the vial via the cannula (131) to the reservoir (134). Retracting the plunger may be via a manual operation of the plunger or by an automated activation of the plunger by attachment of the drive unit (120) to the wearable dispensing unit (130) as described further below.

The method may attach (207) a drive unit to the wearable dispensing unit (130) to provide a delivery device (100). This may automatically release (208) the detachable support from the wearable dispensing unit. Automatically releasing the detachable support from the wearable dispensing unit may be caused by the drive unit engaging with an attaching arrangement attaching the detachable support to the wearable dispensing unit.

The method may expose (209) a skin adhesive surface on a delivery side of the wearable dispensing unit and the tip of the cannula. Releasing the detachable support from the wearable dispensing unit may expose the skin adhesive surface. The method provides (210) the delivery device (100) ready for application to a user's body using the adhesive surface to attach to the user's skin and including insertion of the cannula tip into the user's skin.

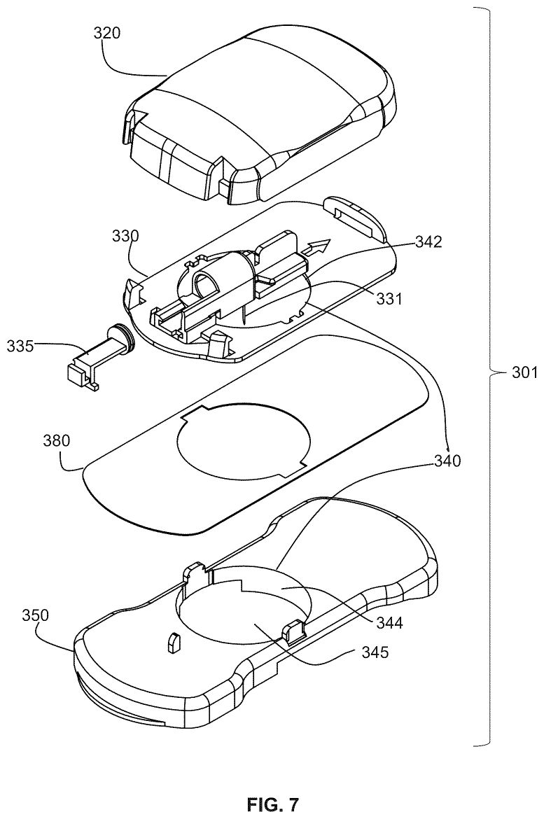

FIG. 7 is an exploded perspective views of another example embodiment of a delivery device assembly (301) including an interface (340) suitable for filling from a vial. The delivery device assembly (301) may include a drive unit (320), a dispensing unit (330), an adhesive member (380), and a detachable support (350). The dispensing unit (330) and the detachable support (350) may together form the interface (340) dimensioned to receive the top of a vial so that a tip (332) of a cannula (331) of the dispensing unit (330) is positioned to pierce a septum of the vial for access to a medicament stored in the vial.

The vial may be a standard 10 ml vial as used for filling pumps or syringes. The vial may have a rubber septum at the top for piercing through with a needle, or in this case with the tip of the cannula. In order to accommodate a vial as opposed to a delivery pen, a larger diameter opening in the interface (340) is needed and heights and needle length adjusted accordingly. Due to different requirements of the needle length for insertion in a vial, the interface (340) may be provided solely on the detachable support (350) in the form of an opening (345) and surrounding collar (344) without a recess in the dispensing unit (330). In other embodiments, the recess (342) in the dispensing unit (330) may be provided around the cannula (331) without need for the detachable support (350). Conversely, no recess (342) may be provided in the dispensing unit (330).

Unlike a pen, the vial does not have a dispensing mechanism, therefore the plunger (335) of the dispensing unit (330) must draw the medicament out from the vial. Typically, to maintain pressure within a vial, a syringe plunger is first retracted to fill the syringe with air, then the needle is pierced through the septum of the vial. The syringe plunger is advanced to dispense air into the vial thereby pressurizing it and then the syringe plunger is retracted to draw liquid out. Optionally, the syringe plunger can be partially depressed and retracted again to clear any remaining air out of the syringe and into the vial. This is the standard method for using a vial.

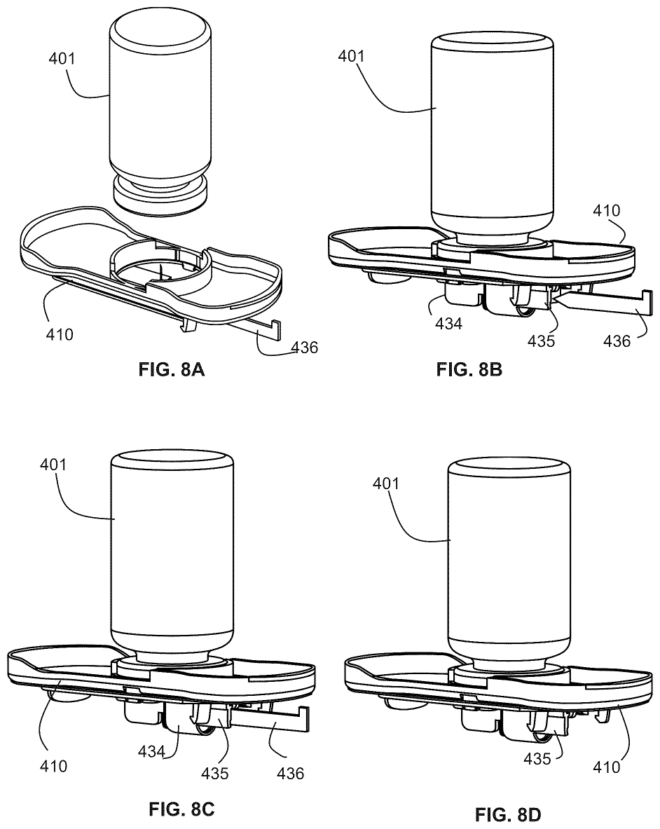

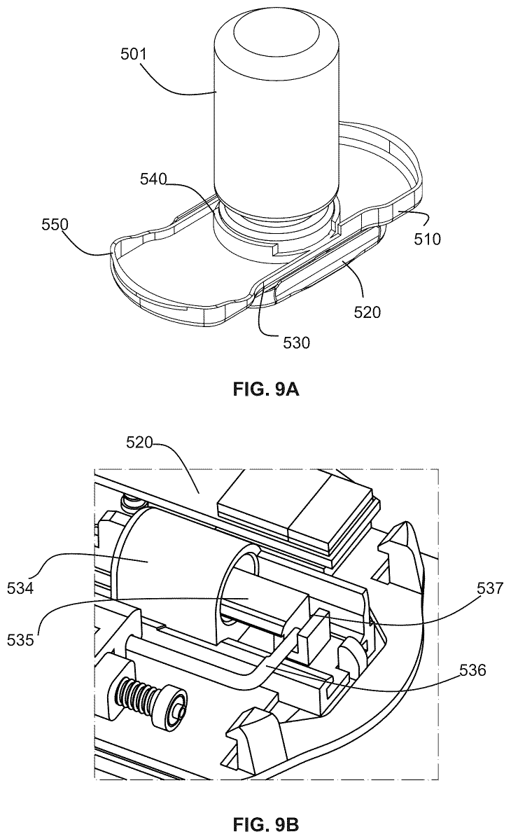

For use of a vial as a filling device for the dispensing unit (330), two example methods are described. A first method is described with reference to FIGS. 8A to 8D and a second method with reference to FIGS. 9A and 9B.

FIGS. 8A to 8D are a sequence of perspective views of an example embodiment of a delivery device component (410) being filled from a vial (401).

In this example embodiment, a user manually actuates the plunger (435) of the dispensing unit (430) forward and back. The plunger (435) may be made large enough to manipulate by hand by having an extension member (436). The extension member (436) may be used as a handle for operating the plunger (435) to fill the reservoir (434). The extension member (436) may be removed by being broken-off, unsnapped, unclipped, or otherwise detached and discarded before a drive unit is attached to the dispensing unit (430).

FIGS. 8A and 8B show the vial (401) being inserted into the interface (440) with the extension member (436) and plunger (435) pulled back. FIG. 8C shows the extension member (436) and plunger (435) pushed forward to expel air and then the extension member (436) and plunger (435) are pulled back to fill the reservoir (434) with medicament. FIG. 8D shows the extension member (436) having been removed.

FIGS. 9A and 9B show another example embodiment of a delivery device component (510) being filled from a vial (501). In this embodiment, the drive unit (520) is used to operate the plunger (535) to draw medicament from the vial (501). FIG. 9A shows a vial (501) inserted into an interface (540) of the detachable support (550) and the dispensing unit (530) with the drive unit (520) attached to the dispensing unit (530). The drive unit (520) may be attached to the dispensing unit (530) before inserting the vial (501) into the interface (540). In this embodiment, the detachable support (550) is not automatically released when the drive unit (520) is attached to the dispensing unit (530).

FIG. 9B shows a detailed view of the inside of the dispensing unit (520) with the reservoir (534) and plunger (535) shown. The view of FIG. 9B also includes a plunger operating member (536) of the drive unit (520) for retraction of the plunger (535) for drawing medicament from the vial (501). The plunger (535) includes a groove (537) for receiving the plunger operating member (536) to allow it to operate the plunger (535) in both directions to retract the plunger (535) when filling the reservoir (534) through the cannula, and to push the plunger (535) when delivering the medicament through the cannula to the user.

The user may attach the drive unit (520), then install the vial (501) in the interface (540), and the drive unit (530) may have a controller that may control a sliding of the plunger (535) forward and back to accomplish filling the reservoir (534) through the cannula. The user may then wait until filling is completed, for example, by monitoring a movement of the plunger (535), watching for an indicator, or waiting for a notification in an associated application provided for the delivery device, before removing the detachable support (550) from the delivery device and wearing the device.

In the embodiment of FIGS. 9A and 9B, the following operation may be carried out. The plunger (535) may be fully retracted in shipment or after user preparation. The drive unit (520) may be snapped-on to the dispensing unit (530) by the user. A vial (501) may be installed into the interface (540) of the delivery device component (510) with the cannula puncturing the septum of the vial (501). A controller of the drive unit (520) may perform a homing process to seat the plunger operating member (536) into the groove (537) of the plunger (535). The controller may advance the plunger (535) fully forward, expelling air from the reservoir (534) into the vial (501). The controller may then retract the plunger (535) (partially or fully) to allow medicament to be drawn from the vial (501) into the reservoir (534). Optionally, the controller partially advances the plunger (535) again to expel any remaining air bubbles. Optionally, the controller again retracts the plunger (535) to the final desired fill volume of the reservoir (534). The user may receive an indication that filling and priming are complete. The indication may be the stopped movement, an indicator light, an application notification, etc. The user may then remove the vial (501) from the interface (540). The user may remove the detachable support (550) to leave the delivery device (500) ready for attachment to the user's skin and insertion of the cannula into the skin.

The controller may have a programmed sequence that controls the plunger movement when the drive unit (520) is first attached to the dispensing unit (530) and then an indication is provided that a vial (501) has been inserted into the interface (540).

In FIGS. 4A to 4E, a first attaching arrangement (160) was shown and described for attaching and detaching the detachable support (150) to and from the dispensing unit (130). Alternative attaching arrangements as now described with reference to FIGS. 10A, 10B, 11, 12A, 12B and 13. Any of the attaching arrangement may be used with either the pen filling interface (140) or the vial filling interface (340, 440, 540).

FIGS. 10A and 10B are top and bottom perspective views of an example embodiment of a delivery device component (610) including a second attaching arrangement (660). In this arrangement, the attaching arrangement (660) may include manual levers (661, 662) for retaining the dispensing unit (630). The manual levers (661, 662) include clip portions (663, 664) clipping over opposing edges of the dispensing unit (630) and the detachable support (650) with the levers (661, 662) extending on the detachable support side. When the drive unit is installed on the dispensing unit (620), the user may manually squeeze the manual levers (661, 662) to detach the clip portions (663, 664) and release the dispensing unit (630) from the detachable support (650):

FIG. 11 is a top perspective views of a further embodiment of the delivery device component (710) including a third attaching arrangement (760). In this embodiment, similar clip portions (763, 764) are provided clipping over opposing edges of the dispensing unit (730) and the detachable support (750). In this embodiment, there are no levers for manual release and when the drive unit is installed, it is pulled upwards away from the detachable support (750) to overcome the snap force of the clip portions (763, 764).

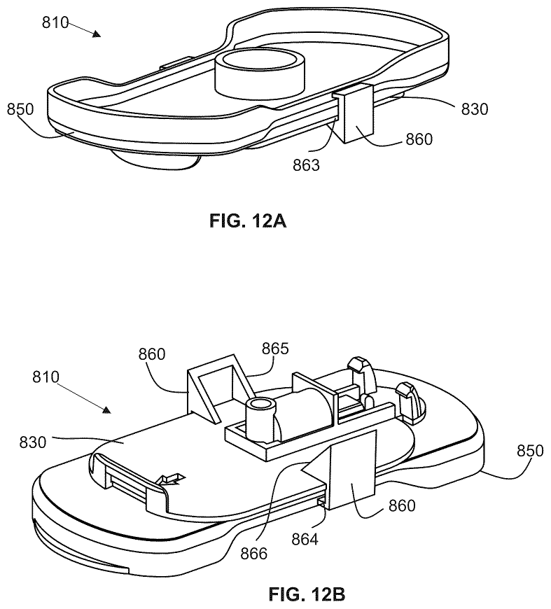

FIGS. 12A and 12B are top and bottom perspective views of a further embodiment of the delivery device component (810) including a fourth attaching arrangement (860). This attaching arrangement (860) includes similar clip portions (863, 864) provided clipping over opposing edges of the dispensing unit (820) and the detachable support (840). In this embodiment, the clip portions (863, 864) have ramps (865, 866) facing upwards from the dispensing unit (830) so that a drive unit (820) being installed on the dispensing unit (830) will force the ramps (865, 866) outwards thereby releasing the clip portions (863, 864). The clip portion (863, 864) may stay attached to the detachable support (850) which is released from the dispensing unit (830). In this way, the attaching arrangement (860) automatically disengages when the drive unit is installed, but this is not related to a filling interface as in the first attaching arrangement (160).

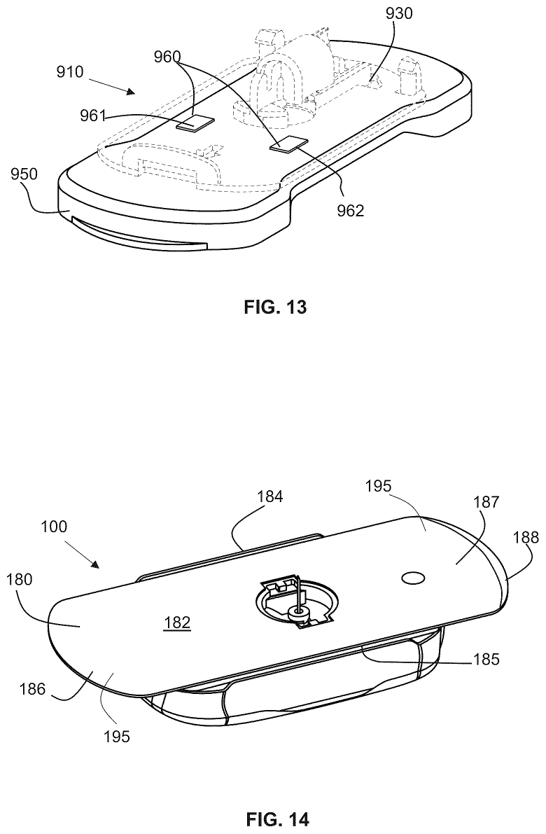

FIG. 13 is a top perspective view of a further embodiment of the delivery device component (910) including a fifth attaching arrangement (960) for attaching the dispensing unit (930) to the detachable support (950). In this embodiment, instead of latches, adhesive patches (961, 962) are provided on corresponding facing surfaces of the dispensing unit (930) and the detachable support (950). The adhesive patches (961, 962) may have enough securement to allow shipping and handling but which then can be overcome by the user pulling upward on an attached drive unit.

FIG. 14 is a perspective underside view of an example embodiment of a delivery device (100) showing an adhesive arrangement of the adhesive member (180). This may be ready for manual insertion of the cannula (131) into the user's skin to the required depth before the adhesive surface (182) is in full contact with the skin surface. The “skirt” (195) is a region of adhesive material that is not attached to the delivery device and that adheres to the skin with flexibility to provide a more secure attachment. The skirt (195) may be within the footprint of the delivery device or may extend beyond the footprint of the delivery device.

The described delivery device (100) includes skirt tabs (186, 187) extending at opposing ends of the delivery device (100). With delivery devices (100) that are intended for wear of less than 24 hours, the skirt does not need to be as secure as other longer-term devices as it is exposed to fewer environmental factors (for example, less physical activity, contact sports, and jostling throughout the day) and needs to adhere for a shorter duration. This means that the adhesion strength can be less and a small amount of peeling at the edges is acceptable. The surface area of the adhesive surface (182) may be reduced commensurate with the wear time of the delivery device (100), for example, less than 24 hours, 12 hours, or 8 hours. An “hourglass” skirt shape may also be used with the skirt tabs being wider than the middle portion of the delivery device (100).

The skin adhesive surface (182) may be provided so that it does not extend beyond the footprint of the delivery device (100) on the opposing sides (184, 185) giving space for a thumb and fingers to grasp the delivery device (100) without having to pinch with fingertips above the adhesive material and eliminating the potential for accidentally folding down the sides of the adhesive material onto itself.

In alternative embodiments, the opposing sides (184, 185) may extend to match the footprint of the delivery device (100) or may extend a small amount (for example, 1 mm or less) beyond the footprint of the delivery device (100). The opposing sides (184, 185) of the skin adhesive member (180) will still not impact a user's finger and thumb gripping the sides of the delivery device (100). This may maximize adhesive area to the skin and may prevent the plastic parts of the delivery device (100) from directly contacting the skin while worn.

There may be a skirt on the sides but it is fully recessed beneath the delivery device (100) in the grip areas. If the wearable delivery device were adhered to the patch out to its edge, it would easily pull the patch off of the skin, because patch adhesion is weak in tension. Instead, the delivery device (100) pulls on the patch inward from the edge, creating a region of shear, which is secure because adhesive patches are very strong in shear.

Retaining exposed skirt tabs (186, 187) at the front and rear of the delivery device (100) maximizes overall adhesion to the skin. The exposed skirt tabs (186, 187) compensate for the recessed side portions of the adhesive in the gripping area. The long axis of the delivery device (100) is the most likely to pull away from the rounded contours of the human body, so securing the front and rear with exposed skirt tabs (186, 187) protects the areas of the wearable most vulnerable to peeling off. Also, by extending adhesive material in the exposed skirt tabs (186, 187), this provides regions that can be taped down with secondary adhesives for users who need this additional adhesion, without having to tape the entire delivery device down. Furthermore, rubbing an adhesive patch against the skin is the best way to promote adhesion of a pressure sensitive adhesive. The delivery device (100) presses down on the adhesive patch beneath it, but the exposed areas of the exposed skirt tabs (186, 187) can be manually rubbed against the skin by the user for maximum adhesion in these most critical areas.

Maximizing adhesion strength along the long axis of the delivery device (100) helps ensure that the delivery device remains attached and does not require a full perimeter of adhesion to stay attached. Even where there is weak adhesion on the sides of the delivery device (100), as long as the front and rear are secure, the delivery device can still stay securely attached.

For shorter duration wear delivery devices, it is important that removal is easy, trauma-free, and results in minimal skin irritation and user annoyance. A small lip of non-adhesive area (188) may be provided at the end of one of the exposed skirt tabs (186, 187) to act as a peel-tab for a user during removal. This retains an accessible area of the patch exposed beyond the footprint of the wearable providing an easy-removal tab.

FIGS. 15A, 15B, and 15C show an example embodiment of a delivery device component (290) showing another example of an adhesive arrangement that may be used with any of the described embodiments or with other forms of wearable medicament delivery devices. FIG. 15A shows an exploded perspective view of the delivery device component (290) including the dispensing unit (130) as previously described with an example embodiment of an adhesive arrangement (280) and a detachable support (250).

The adhesive arrangement (280) may include a skin adhesive member (281) that is attached to the delivery side of the dispensing unit (130) and has a skin adhesive surface (282) for attachment to a user's skin. The skin adhesive surface (282) is configured to be exposed when the detachable support (250) is removed. The skin adhesive member (281) includes a skirt portion (295) that flexible with respect to the wearable dispending unit (130) to provide flexible attachment to a user's skin.

A device adhesive layer (284) in the form of an area of double-sided pressure sensitive adhesive (PSA) material may be provided and adhered to the skin adhesive member (281) on a surface facing the dispensing unit (130) with the other side of the PSA portion being provided for adhesion to the dispensing unit (130) during manufacture of the device. In this embodiment, the PSA portion has a width (285) and length (286), with at least the width (285) being less than the width of the dispensing unit (130). In this way, the PSA portion (284) is within the footprint (139) of the dispensing unit (130). The reduced width (285) of the PSA portion allows the side portions of the adhesive surface (282) to form skirt portions that are flexible for adhesive to the user's skin whilst being protected underneath the dispensing unit (130). The areas of the skin adhesive member (281) that surrounds the PSA portion form a skirt that provides flexibility for attachment to the user's skin with an upper surface having a protective surface for exposure during use. An alternative embodiment of the PSA portion is described below with reference to FIG. 16A in which some of the PSA portion may extend into the skirt area.

The adhesive member (281) (and therefore the skirt) may all be within the footprint (139) of the dispensing unit (130) or may have one or more portions extending beyond the footprint (139). For example, one or more end portions of the skin adhesive member (281) may extend from the dispensing device footprint (139) whilst the side portions are within the footprint. The adhesive member (281) may have a lip (288) providing a non-adhesive area at one end of the adhesive surface (282) to facilitate removal of the adhesive member (281) from the user's skin at the end of the use.

The adhesive arrangement (280) of this embodiment may include a backing layer (270) configured to cover the adhesive surface (282) prior to adhesion to a user's skin. The backing layer (270) may have at least one attachment portion for attachment to the detachable support (250) and configured such that removal of the wearable dispensing unit (130) from the detachable support (250) peels the backing layer (270) from the adhesive surface (281).

The dispensing unit (130) has a fixed cannula that protrudes from the reservoir through the adhesive arrangement (280), and this may be a needle stick hazard if a user has to manually reach underneath to peel off an adhesive backing layer (270). Therefore, the backing layer (270) is attached to the detachable support (250) so that when the user lifts the dispensing unit (130) off the detachable support (250) (as shown in FIGS. 15B, 15C and 15D) and exposes the cannula, which has previously been guarded by the detachable support (250). The backing layer (270) peels automatically without the user needing to put their fingers near the cannula. This also reduces overall handling complexity and number of steps for the user to perform.

In the embodiment shown in FIGS. 15A to 15C, the backing layer (270) is a butterfly liner formed of two parts (272, 273), each part including an adhesive covering portion (274, 275) and an attachment portion (276, 277) with the attachment portions (276, 277) folded back on the adhesive covering portions (274, 275) and extending from a central area (283) of the adhesive surface (282). In one embodiment, each of the two parts (272, 273) of the butterfly liner may be folded back on itself such that the attachment portion (276, 277) is generally the same size as the adhesive covering portions (274, 275). The two parts (272, 273) may overlap (278) at a line at the central area (283) and the folded parts (272, 273) may accommodate the overlap with angled folds.

In the embodiment shown in FIG. 15D, the two parts (272A, 273A) have a different arrangement of the attachment portions (276A, 277A) with one of the attachments portions (277A) folded back and the other attachment portion (276A) being a flat extension. In other embodiments, the backing layer may be a single piece with an attachment portion to the detachable support. In one embodiment, there may be a single liner in the form of a single butterfly wing having an adhesive covering portion covering the whole skin adhesive surface and an attachment portion folded back on the adhesive covering portion.

The detachable support (250) of the embodiments of FIGS. 15A to 15D includes at least one liner anchor member (251, 252) that projects from one or each end a device facing surface (253) of the detachable support (250). The anchor member(s) (251, 252) may be hooks or barb shapes. The liner anchor members (251, 252) are configured to engage with the attachment portions (276, 277) of the backing layer. In the embodiment of FIGS. 15A to 15C, there are a pair of anchor members (251, 252) at each end portion of the detachable support (250). In an alternative embodiment, in which a backing layer is a single part with a folded extension, a single anchor member or a single pair of anchor members may be provided. In the embodiment of FIG. 15D, only one set of anchor members (252A) is required onto which both attachment portions (276A, 277A) are attached. Both attachment portions (276A, 277A) are anchored to the detachable support (250) at one or more anchor members (252A).

The device facing surface (253) of the detachable support (250) may have one or more recessed areas (254) to accommodate the liner anchor member(s) (251, 252). In the embodiments of FIGS. 15A to 15C, a recessed area (254) is provided at each end of the detachable support (250) with the recessed area (254) having a sloping portion (256) sloping at an angle from a central portion (257) to flat end portions (255) to form a bridge shape. The bridge shape provides the central portion (257) to abut the dispensing unit (130) when the detachable support (250) is attached whilst providing a space between the adhesive member (281) and the flat end portions (255) that support the anchor members (251, 252) allowing the attachment portions (276, 277) of the backing liner (270) to be held away from the adhesive covering portions (274, 275). In FIG. 15D, two recesses (254) are shown, however the anchor member (251) and associated recess (254) may be replaced by an extension of the flat central portion (257) of the detachable support (250).

The attachment portions (276, 277, 276A, 277A) include at least one slit (266, 267) configured for attachment to the anchor member(s) (251, 252). The slits (266, 267) may be formed through all layers of the adhesive arrangement (280) for ease of manufacture, including the skin adhesive member (280) and the layers of the backing liner (270). The slits (266, 267) may be in-line with the direction of removal of the backing layers and/or in-line with the direction the user peels the skin adhesive member (281) off their skin so that the slits do not expand. In this example, the direction of the removal of the backing layers is the same as that of the direction of removal from the skin; however, this does not need to be the case. Slits (266, 267) are used as they stay closed and do not expand in the skin adhesive member (281).

A constraint in patch die-cutting is that the same cut pattern should pass through all of the layers of material, otherwise more costly and specialized processes are required. Therefore, thin slits (266, 267) are cut at each end, through all layers. The material may be sliced with the die with no material removed, so the adhesion and cosmetics of the skin adhesive layer are minimally affected, if at all.

The butterfly folds of the extension portions (276, 277) of the backing layer (270) are pressed onto the anchor members (251, 252) of the detachable support (250). Unlike circular cutouts, which would have to be hooked onto the plastic or retained with a secondary heat-staking process, the elongated slits can expand to the sides around the anchor members (251, 252), but retain a tight grip once pressed on. This also requires no secondary processes (sliding material laterally onto a latch, gluing, heat staking, etc.) and is achieved in a simple top-down assembly motion, ideal for efficient automated assembly.

The slits (266, 267) and anchor members (251, 252) may be positioned below the footprint of the wearable delivery device (100), such that the skin adhesive member (281) may not necessarily extend beyond this footprint.

Other example embodiments of additional aspects are described with reference to FIGS. 16A to 16H. In FIG. 16A, an adhesive arrangement (1280) is shown including a device adhesive layer (1284) attached to a device facing surface of a skin adhesive member (1281) and a backing liner (1270) that protects a skin adhesive surface of the skin adhesive member (1281). The device adhesive layer (1284) is formed of a pressure sensitive adhesive (PSA) material and, in this embodiment, extends to opposing side edges (1285, 1286) of the skin adhesive member (1281). The width of the adhesive arrangement (1280) may be the same or less than the width of the wearable delivery device. The skin adhesive member (1281) has a skirt portion (1220) with opposing side skirts (1221, 1222) that are within the footprint and the device adhesive layer (1284) extends across the side skirts (1221, 1222).

FIG. 16B shows an example embodiment of the detachable support (1250) that is configured to apply pressure to the area of the device adhesive layer (1284) excluding areas of the side skirts (1221, 1222) to which the device adhesive layer extends. This configuration includes a flat planar area (1253) that exerts a pressure on the device adhesive layer (1284) with the flat planar area (1253) having cut-away sides (1254, 1255) that correspond to the side skirts (1221, 1222) such that no pressure is applied in these areas.

The device-facing device adhesive layer (1284) should not be left to stick on the user's skin, clothing, bedding, etc. so it would need to be covered by the dispensing unit (1230) when attached. The skin adhesive member (1281) should be pressed firmly down against the user's skin everywhere, including these sides, so the dispensing unit (2130) may extend downward to press the skin adhesive member (1281) against the user's skin.

FIGS. 16C to 16H show different embodiments of a dispensing unit (1230) that includes side wings (1231, 1232). Each of FIGS. 16C to 16H shows an underside view and a top view of the dispensing unit (1230). The side wings (1231, 1232) may be configured to not stick to the device adhesive layer (1284) at the areas of the side skirts (1221, 1222) (FIGS. 16C to 16E). The side wings (1231, 1232) may be configured to flex with the side skirts (FIGS. 16F to 16H).

In the embodiments shown in FIG. 15A, an “island” of double-sided PSA is used for the device adhesive layer (284) and placed in the middle of the skin adhesive member (281) to adhere to the dispensing unit (130). An island is used so that the device adhesive layer (284) does not adhere the outer edges of the dispensing unit (130) to provide an unattached skirt (195) for good adhesion to the user's skin, so that the adhesive pulls on the skin in shear and not in tension as tape adhesion is strong in shear and weak in tension.

There is a manufacturing cost involved with die-cutting a piece of PSA separately as an island and including a process to place it accurately onto the patch. The tolerance of placement can also vary (for example +/−0.75 mm), so the design must be robust to this misalignment relative to the patch and reservoir, this is especially the case with a very small skirt on the sides as described. An alternative embodiment shown in FIG. 16A provides is a continuous strip of double-sided PSA as the device adhesive layer (1284) along the center of the skin adhesive member (1281). This arrangement does not require separate die-cutting and placement processes and avoids placement tolerance on the sides.

To form the side skirts (1221, 1222) which form good adhesion to skin, the dispensing unit (1230) has side wings (1231, 1232) that do not adhere or not adhere well to these side skirts (1221, 1222) of the device adhesive layer (1284) in these regions of the underside of the dispensing unit (1230). Alternatively, the side wings (1231, 1232) do adhere to the side skirts (1221, 1222) of the device adhesive layer (1284) but flex with the device adhesive layer (1284).

FIGS. 16C to 16E show three example embodiments of a dispensing unit (1230A, 1230B, 1230C) that have side wings (1231, 1232) with different methods of preventing strong adhesion.

In FIG. 16C, the side wings (1231A, 1232A) have cut-outs (1233) to minimize adhesion to the device adhesive layer (1284). In FIG. 17D and 17E, the side wings (1231B, 1232B, 1231C, 1232C) are textured, such as with small dots (FIG. 17E) or ribs (FIG. 17D), such that the device adhesive layer (1284) does not adhere well or only adheres in a small portion of its surface area so that it peels away these areas easily. It is preferred that ribs (FIG. 17D) do not connect to the center area (1234) so that when the device adhesive layer (1284) peels off of them, it does not continue peeling into the center area (1234) of desired adhesion.

FIGS. 16F to 16G show three examples of embodiments of a dispensing unit (1230D, 1230E, 1230F) with side wings (1231, 1232) being flexible so that even if they remain attached to the device adhesive layer (1284) and therefore the skin adhesive member (1281), they can flex easily and function as part of the skirt. This could be achieved by making the material very thin and/or with cutouts to minimize its rigidity. FIG. 16F shows side wings (1231D, 1232D) with cut-outs (1241). FIG. 16G shows side wings (1231E, 1232E) with multiple flaps (1242). FIG. 16H shows side wings (1231F, 1232F) with single flaps (1243).

In conjunction with the embodiments of FIGS. 16A to 16H, during manufacturing the device adhesive layer (1284) may be pressed firmly against the dispensing unit (1230) in the center area to promote adhesion whilst the skirt areas would not be pressed against the dispensing unit (1230). The detachable support (1250) may be configured so that it does not press against the dispensing unit (1230) in the skirt areas after manufacturing assembly and during storage. The device adhesive layer (1230) may be formed of PSA (Pressure Sensitive Adhesive) material that is pressure sensitive and so adhesion can be reduced by not applying pressure in these side skirt areas.

A detachable support for a wearable medicament delivery device may be provided for a medicament delivery device that is adhered to a user's skin. The medicament delivery device may be as described herein or may be another form of skin adhesive device. The detachable support may be in the form of a protective base for the delivery device attached when packaged before use. The detachable support may include a central planar area configured to conform with a portion of an underside of the delivery device corresponding to an area of a device adhesive layer between a dispensing unit and a skin adhesive layer of the delivery device, where the planar member exerts pressure on the device adhesive material during packaging. The detachable support may include an adhesive protection removal arrangement configured to expose the skin adhesive surface of the skin adhesive layer of the delivery device on removal of the delivery device from the detachable support. This may be by simple removal of the detachable support that exposes the skin adhesive surface or by effecting the removal of an adhesive liner from the skin adhesive surface. The central planar area may not extend across the full width of the delivery device so that it does not exert a pressure on a skirt area of the skin adhesive member that may have adhesive on its device-facing side as well.

An adhesive arrangement may be provided for a medicament delivery device that is adhered to a user's skin. The medicament delivery device may be as described herein or may be another form of skin adhesive device. The adhesive arrangement includes: a skin adhesive member configured to form a flexible skirt extending from an underside of the delivery device and having a skin adhesive surface: a device adhesive layer provided on an upper surface of skin adhesive member and for attachment to an underside of delivery device; and a backing liner covering the skin adhesive surface and configured for removal on release of the delivery device from a detachable support.

A method of manufacture of an adhesive arrangement of a medicament delivery device may include the following steps. A skin adhesive member may be provided and a native backing may be removed from an adhesive surface of the skin adhesive member. A backing liner may be attached to the adhesive surface of the skin adhesive member. A device adhesive layer may be provided having dimensions covering a width of the skin adhesive member and less than a length of the skin adhesive member. A native backing may be removed off the device adhesive layer, and the device adhesive layer applied to a device-facing surface of the skin adhesive member. Once this stack is formed, the stack may be cut to shape as a combined stack of the skin adhesive member, backing liner, and device adhesive layer. The extension of the device adhesive layer to the width of the skin adhesive member removes the need to die-cut the device adhesive layer separately.

One or more anchor openings may be cut through at least one end portion of the combined stack. The anchor openings may be cut as slits in-line with the direction of removal of the backing liner and/or the direction of removal from the user's skin after use to prevent opening of the slits. The slits are only used in the backing liner but forming them through the combined stack improves manufacturing efficiency.

The method of manufacture may include applying the adhesive arrangement to a dispensing unit by removing a second native backing off the device adhesive layer and applying the device adhesive layer applied to an underside of the dispensing unit. The method may include applying pressure from a detachable support that is attached to the dispensing unit during manufacture and for packaging such that the adhesive arrangement is sandwiched between the dispensing unit and the detachable support. The detachable support may only apply pressure to a central region of the device adhesive layer that does not extend over a skirt portion.

FIGS. 17A and 17B are views of another embodiment of the detachable support (2150). In this embodiment, an attaching arrangement (2160) is provided at the interface (2140) between the medication filling device and the dispensing unit that includes at least one resilient portion (2171, 2172). The at least one resilient portion (2171, 2172) is provided in addition to the latches (2161, 2162) that are previously described. In the embodiment shown, there are opposing resilient portions (2171, 2172) between the latches (2161, 2162). The two opposing resilient portions (2171, 2172) are curved as part of the receiving collar (2144) for receiving the medication filling device.

The resilient portions (2171, 2172) are configured to press against and stabilize the medication filling device when inserted in the interface (2140). The opposing resilient portions (2171, 2172) provide slight tactile feedback when installing the medication filling device into the interface and holds it steady during filling. The opposing resilient portions (2171, 2172) at the interface may together be configured to grip the medication filling device such that the detachable support (2150) remains attached to the medication filling device together with the dispensing unit and detached from packaging. This adaptation of the attaching arrangement may be combined with any of the previous embodiments of the detachable support.

FIGS. 17A and 17B also show other variations of the detachable support (2150) including the receiving collar (2144) being provided on the dispensing unit facing side (2151) provided a smooth outward facing side (2152) of the detachable support (2150).

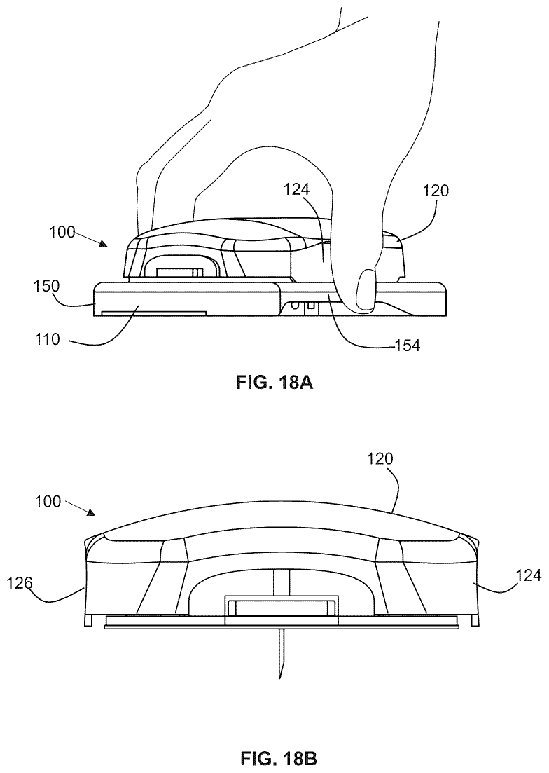

FIGS. 18A and 18B are views showing a gripping arrangement of the delivery device component (110) and the delivery device (100) in FIG. 18A and only the delivery device (100) in FIG. 18B. The drive unit (120) may have a form having opposing side portions (124, 126) of a depth for gripping by a human finger and thumb. The detachable support (150) may have similar gripping side portions (154).

The overall shape of the wearable delivery device (100) may be smooth and rounded to avoid snagging, especially in the front and rear of the long axis where the delivery device (100) may be most prone to pulling away from curved body parts. The drive unit (120) may completely cover the top and sides of the dispensing unit proving a smooth outer surface. The opposing side portions (124, 126) of the drive unit (120) may have vertical or concave regions that enable stable gripping. Other designs may include ridges, lips, texture, or other features to improve gripping strength and stability. The concave region may be provided only in the center of the sides for gripping and is smoothed out and rounded at the corners to avoid snag points or uncomfortable sharp corners.

The aspect ratio of width to height is important for grasping the delivery device (100) in a stable manner. The delivery device (100) has the dispensing unit (130) that sits below the drive unit (120), so to maximize grip height of the exterior controller, the sides of the drive unit (120) may extend downward to the level of the skin, surrounding the dispensing unit (130) below it.

The detachable support (150) elevates the drive unit (120) off a supporting surface, giving room for fingers to naturally grip the drive unit (120) with the pads of the fingers rather than the fingertips, allowing for a more secure grip.

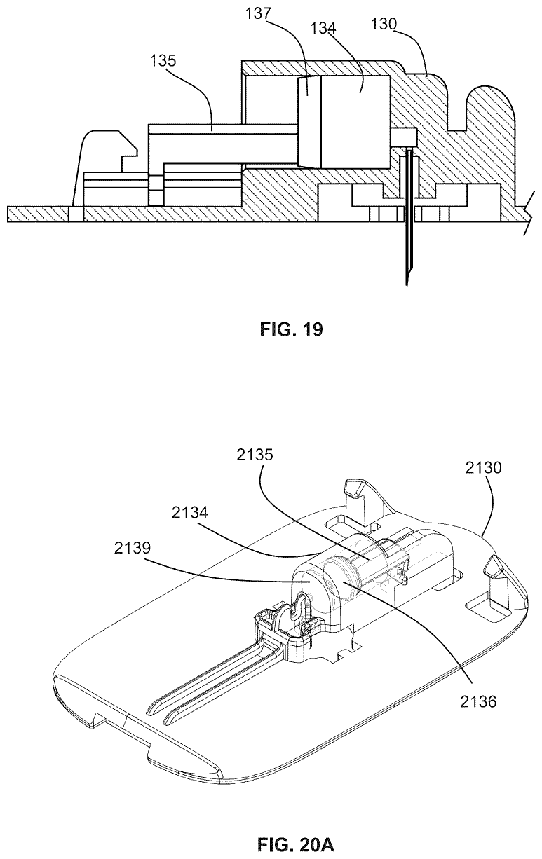

FIG. 19 is a cross-section of an embodiment of a delivery device component of any of the previous embodiments including a plunger sealing arrangement. The dispensing unit (130) includes a plunger (135) for movement within the reservoir (134) with the plunger (135) having an interference fit (137) with an internal surface of the reservoir (134) removing a need for an elastomeric sealing member such as an o-ring. The interference fit (137) is achieved by one or both of the materials of an edge of the plunger (135) and the internal surface of the reservoir (134) being somewhat compliant so that the parts can be assembled and slide together with a liquid tight fit. The materials may be of polypropylene or polyethylene.

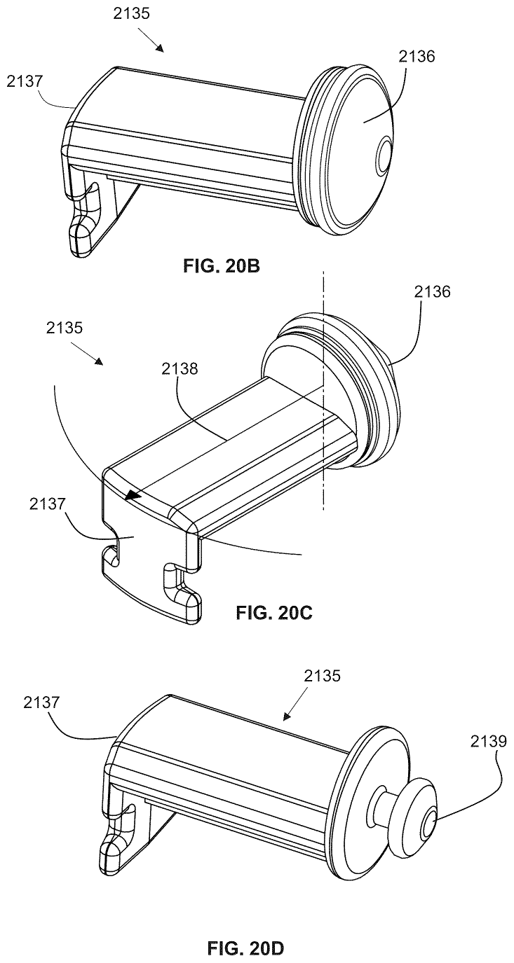

FIGS. 20A to 20D show a further embodiment of a plunger (2135) and sealing arrangement for interaction with the reservoir (2134) of the dispensing unit (2130). The plunger (2135) of this embodiment includes a full-face seal (2136) that covers the entire front end of the plunger (2135). The full-face seal (2136) has the advantage that it is easier for a human or automation equipment to press this seal onto the front of the plunger compared to having to carefully stretch an o-ring and position it within a groove. The full-face seal (2136) has a front elastomeric face that conforms to a front (2139) of the reservoir (2134) leaving less dead volume when empty and minimizes air in the reservoir (2134) after filling. The full-face seal (2136) may be of a curved conical shape.

The full-face seal (2136) also isolates the plunger (2135) from drug contact, allowing more material and colorant options for the plunger to optimize for cost, performance, and visibility without having to be medication compatible. The plunger may be a contrasting color to the reservoir that may be transparent so that plunger movement and fill level are visually apparent to a user. FIG. 20D shows the plunger (2135) without the full-face seal (2136) and having a protrusion (2139) for holding the seal (2136) in place.

The plunger (2135) may have a rounded rear face (2137) rounded about a longitudinal axis (2138) of the plunger (2135). The full-face seal (2136) fits tightly in the reservoir bore, but the rear face (2137) of the plunger (2135) may have some lateral clearance. When the rear face is flat, rotation of the plunger affects advancement of the seal, which creates inconsistent backlash when priming the device. By rounding the rear face (2137) so all points on the rear face are equidistant from the seal, backlash related to lateral shifting of the plunger is eliminated. A goal is for the user to wear the device without having to manually prime liquid onto the ground to take up backlash, so making backlash as minimal and consistent as possible is important.

The foregoing description has been presented for the purpose of illustration; it is not intended to be exhaustive or to limit the invention to the precise forms disclosed. Persons skilled in the relevant art can appreciate that many modifications and variations are possible in light of the above disclosure. In particular, features shown in one of the embodiments may be used in combination with one or more features of the other embodiments.

The language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention.

Finally, throughout the specification and accompanying claims, unless the context requires otherwise, the word ‘comprise’ or variations such as ‘comprises’ or ‘comprising’ will be understood to imply the inclusion of a stated integer or group of integers but not the exclusion of any other integer or group of integers.

1. A medication fluid delivery device component, comprising:
a wearable dispensing unit having a cannula in fluid communication with a reservoir for delivery of a medication fluid from the reservoir via a tip of the cannula during use of the dispensing unit; and
an interface disposed at least partially around the cannula on a delivery side of the wearable dispensing unit and the interface being configured to receive an end portion of a medication filling device for filling of the reservoir via the tip of the cannula.

2. The delivery device component of claim 1, including a detachable support for the wearable dispensing unit with at least a portion of the interface being provided on the detachable support and the portion of the interface on the detachable support providing a protective cover for the tip of the cannula.

3. The delivery device component of claim 2, wherein the at least a portion of the interface is a receiving collar surrounding an opening provided in the detachable support.

4. The delivery device component of claim 2, wherein the interface is formed of the portion provided on a detachable support and a recess in the delivery side of the wearable dispensing unit surrounding the cannula.

5. The delivery device component of claim 4, wherein a depth of the recess is a difference between a first length of the cannula required for filling and a second length of the cannula required for insertion into a body.

6. The delivery device component of claim 2, including an attaching arrangement for securing the detachable support to the wearable dispensing unit, wherein the attaching arrangement is disposed at the interface and is configured to have latches that are prevented from release by the end portion of the medication filling device when inserted in the interface.

7. The delivery device component of claim 6, wherein the attaching arrangement is configured to be released automatically by interaction with a drive unit when the drive unit is attached to the wearable dispensing unit.

8. The delivery device component of claim 2, wherein the detachable support includes an engaging protrusion for engaging with a plunger of the wearable dispensing unit to provide a restriction on a movement of the plunger.

9. The delivery device component of claim 2, including an adhesive arrangement including a skin adhesive member disposed on the delivery side of the wearable dispensing unit providing a skin adhesive surface configured to be exposed when the detachable support is removed.

10. The delivery device component of claim 9, wherein the skin adhesive member is within a footprint of the delivery side of the wearable dispensing unit on two opposing sides and extending beyond the footprint at one or both opposing ends.

11. The delivery device component of claim 9, wherein the skin adhesive member includes a backing layer configured to cover the skin adhesive surface and the backing layer having at least one attachment portion for attachment to the detachable support and configured such that removal of the wearable dispensing unit from the detachable support peels the backing layer from the skin adhesive surface.

12. The delivery device component of claim 11, wherein the backing layer is a butterfly liner formed of two parts, each part including an adhesive covering portion and an attachment portion, wherein one or both of the attachment portions are folded back on the adhesive covering portions.

13. The delivery device component of claim 11, wherein the detachable support includes a liner anchor member extending from a device facing surface of the detachable support with the liner anchor member configured to engage an attachment portion of the backing layer and wherein the device facing surface of the detachable support has a recessed area accommodating the liner anchor member.

14. The delivery device component of claim 12, wherein the attachment portion includes at least one slit configured for attachment to the anchor member, wherein the slit is in-line with a direction of removal of the backing layer.

15. The delivery device component of claim 10, wherein the adhesive arrangement includes a device adhesive layer attached to a device facing surface of the skin adhesive member, wherein the device adhesive layer is formed of a pressure sensitive adhesive material and extends to opposing side edges of the skin adhesive member.

16. The delivery device component of claim 15, wherein the skin adhesive member includes a skirt portion that is flexible with respect to the wearable dispensing unit to provide flexible attachment to a user's skin and the skirt portion has opposing side skirts that are within the footprint and the device adhesive layer extends to the side skirts.

17. The delivery device component of claim 16, wherein the detachable support is configured to apply pressure to the area of the device adhesive layer excluding areas of the side skirts to which the device adhesive layer extends.

18. The delivery device component of claim 16, wherein the dispensing unit includes side wings that are configured to not stick to the device adhesive layer at the areas of the side skirts.

19. The delivery device component of claim 16, wherein the dispensing unit includes side wings that are configured to flex with the side skirts.

20. The delivery device component of claim 2, including a surrounding packaging of the wearable dispensing unit, wherein at least part of the surrounding packaging is integrated with the detachable support and wherein a part of the surrounding packaging provides a stable tray during a filling of the wearable dispensing unit.

21. A medication fluid delivery device including:
a medication fluid delivery device component, comprising:
a wearable dispensing unit having a cannula in fluid communication with a reservoir for delivery of a medication fluid from the reservoir via a tip of the cannula during use of the dispensing unit; and
an interface disposed at least partially around the cannula on a delivery side of the wearable dispensing unit and the interface being configured to receive an end portion of a medication filling device for filling of the reservoir via the tip of the cannula; and
a reusable drive unit for connection to the wearable dispensing unit for driving the delivery of the medication fluid from the reservoir through the cannula.

22. A method of use of a medication fluid delivery device, comprising:
filing a wearable dispensing unit through a tip of a cannula, wherein the cannula is in fluid communication with a reservoir for delivery of a medication fluid from the reservoir via the tip of the cannula during use of the dispensing unit.

23. The method of claim 22, including:
positioning a medication filling device in an interface disposed at least partially around the cannula on a delivery side of the wearable dispensing unit such that the cannula is inserted into the medication filling device.

24. The method of claim 22, wherein the medication filling device is an insulin pen and the method includes:
dispensing the medication from the insulin pen via the cannula to the reservoir.

25. The method of claim 22, wherein the medication filling device is a vial and the method includes:
retracting a plunger of the wearable dispensing unit to draw the medication from the vial via the cannula to the reservoir.

26. The method of claim 25, wherein retracting the plunger is via an automated activation of the plunger by attachment of a drive unit to the wearable dispensing unit.

27. The method of claim 22, wherein at least a portion of the interface is provided on a detachable support for the wearable dispensing unit, the method includes:
attaching a drive unit to the wearable dispensing unit thereby automatically releasing the detachable support from the wearable dispensing unit.

28. The method as claimed in claim 27, wherein automatically releasing the detachable support from the wearable dispensing unit is caused by the drive unit engaging with an attaching arrangement attaching the detachable support to the wearable dispensing unit.

29. The method of claim 27, wherein releasing the detachable support from the wearable dispensing unit exposes an adhesive surface on a delivery side of the wearable dispensing unit and the tip of the cannula.

30. The method of claim 22, including:
providing the wearable dispensing unit in a surrounding packaging, at least a portion of the surrounding packaging providing support for the wearable dispensing unit during filling of the wearable dispensing unit.
